SEQUENCE LISTING (20 sequences)

SEQ ID NO: 1 - DNA, 19 bp, Artificial Sequence
  Human insulin forward primer
ccttgtgaaccaacacctg

SEQ ID NO: 2 - DNA, 20 bp, Artificial Sequence
  Human insulin reverse primer
gtagaagaagcctcgttccc

SEQ ID NO: 3 - DNA, 22 bp, Artificial Sequence
  Human glucagon forward primer
cccaagattttgtgcagtggtt

SEQ ID NO: 4 - DNA, 24 bp, Artificial Sequence
  Human glucagon reverse primer
cagcatgtctctcaaattcatcgt

SEQ ID NO: 5 - DNA, 20 bp, Artificial Sequence
  Human somatostatin forward primer
gatgccctggaacctgaaga

SEQ ID NO: 6 - DNA, 22 bp, Artificial Sequence
  Human somatostatin reverse primer
ccgggtttgagttagcagatct

SEQ ID NO: 7 - DNA, 21 bp, Artificial Sequence
  Human FXYD2gamma a forward primer
actgggttgtcgatggacggt

SEQ ID NO: 8 - DNA, 21 bp, Artificial Sequence
  Human FXYD2gamma a reverse primer
cggctcatcttcattgatttg

SEQ ID NO: 9 - DNA, 15 bp, Artificial Sequence
  Human FXYD2gamma b forward primer
gacaggtggtacctg

SEQ ID NO: 10 - DNA, 21 bp, Artificial Sequence
  Human FXYD2gamma b reverse primer
cggctcatcttcattgatttg

SEQ ID NO: 11 - DNA, 20 bp, Artificial Sequence
  Human beta-actin forward primer
ctgtacgccaacacagtgct

SEQ ID NO: 12 - DNA, 20 bp, Artificial Sequence
  Human beta-actin reverse primer
gctcaggaggagcaatgatc

SEQ ID NO: 13 - DNA, 20 bp, Artificial Sequence
  hSLC2A2 Forward primer
atccaaactggaaggaaccc

SEQ ID NO: 14 - DNA, 20 bp, Artificial Sequence
  hSLC2A2 Reverse primer
catgtgccacactcacacaa

SEQ ID NO: 15 - DNA, 20 bp, Artificial Sequence
  hINSULIN Forward primer
aggccatcaagcagatcact

SEQ ID NO: 16 - DNA, 20 bp, Artificial Sequence
  hINSULIN Reverse primer
gcacaggtgttggttcacaa

SEQ ID NO: 17 - DNA, 20 bp, Artificial Sequence
  hPDX1 Forward primer
ccttgtgctcgggttatgtt

SEQ ID NO: 18 - DNA, 20 bp, Artificial Sequence
  hPDX1 Reverse primer
atcatcccactgccagaaag

SEQ ID NO: 19 - DNA, 20 bp, Artificial Sequence
  hVEGF Forward primer
ctacctccaccatgccaagt

SEQ ID NO: 20 - DNA, 20 bp, Artificial Sequence
  hVEGF Reverse primer
gcagtagctgcgctgataga

(19) United States
(12) Patent Application Publication
(10) Pub. No.: US 2024/0226179 A1
(43) Pub. Date: Jul. 11, 2024

(54) TISSUE CULTURE MEDIA FOR SURVIVAL AND ENGRAFTMENT OF HUMAN PANCREATIC ISLETS

(71) Applicant: THE TRUSTEES OF THE UNIVERSITY OF PENNSYLVANIA, Philadelphia, PA (US)

(72) Inventors: Ali NAJI, North Wales, PA (US); Ming YU, Wallingford, PA (US)

(73) Assignee: THE TRUSTEES OF THE UNIVERSITY OF PENNSYLVANIA, Philadelphia, PA (US)

(21) Appl. No.: 17/801,959

(22) PCT Filed: Mar. 1, 2021
(86) PCT No.: PCT/US2021/020301

§ 371 Date: Aug. 24, 2022

Related U.S. Application Data
(60) Provisional application No. 62/983,120, filed on Feb. 28, 2020.

(51) Int. Cl. A61K 35/39; A61K 31/198; A61K 33/00; A61K 38/38; A61K 38/39; A61P 3/10; C12N 5/00; C12N 5/071

(52) U.S. Cl. CPC A61K 35/39; A61K 31/198; A61K 33/00; A61K 38/38; A61K 38/39; A61P 3/10; C12N 5/0018; C12N 5/0677

(57) ABSTRACT

This disclosure provides a culture media that facilitates survival and/or engraftment of transplanted cells. In one embodiment, this culture media comprises collagen I, albumin, L-glutamine and NaHCO₃. In one embodiment, this culture media promotes enhanced survival and engraftment of β-cells of human pancreatic islets transplanted under the skin. The protective effect of this culture media is mediated, at least partly, by upregulating anti-apoptotic signaling pathways.

### CROSS-REFERENCE TO RELATED APPLICATIONS

This application claims priority to U.S. Provisional Application No. 62/983,120, filed Feb. 28, 2020, which is hereby incorporated by reference in its entirety.

### STATEMENT REGARDING FEDERALLY SPONSORED RESEARCH

This invention was made with government support under Grant Numbers DK070430, AI-102430, and DK112217 awarded by the National Institutes of Health. The United States has certain rights in the invention.

### FIELD OF THE INVENTION

This disclosure relates in general to the field of cellular transplantation and cell-based therapies. In one embodiment, there is provided a tissue culture media (TCM) that would allow successful survival and/or engraftment of human islet cells.

### BACKGROUND OF THE INVENTION

Type I diabetes (T1D) is a chronic autoimmune disease in which the insulin-producing β-cells of the pancreatic islets are unable to provide enough insulin to the body to keep blood sugar levels in the normal range. Symptoms of high blood sugars may include increased thirst and urination, weight loss, blurry vision, hunger and weakness. A common therapy for T1D is daily insulin injections to control blood glucose levels. Unfortunately, despite exogenous insulin therapy many patients experience markedly dysregulated glucose levels that predisposes them to the development of secondary complications of the disease, such as early mortality, blindness, renal failure, coronary artery disease and amputation.

Pancreatic islet transplantation has emerged as the most specific β-cell replacement therapy to achieve precise glycemic control in patients with T1D. Currently, the liver is the preferred site for clinical pancreatic islet transplantation. A phase III clinical trial of islet transplantation, in which islets were infused into the recipients via a percutaneous transhepatic approach that involved puncturing the skin and the liver, has confirmed the efficacy of this innovative therapy to render the islet transplant recipients insulin independent and free of life threatening hypoglycemic unawareness. The percutaneous trans-hepatic approach to access the portal circulation, however, is fraught with complications, including portal vein thrombosis, inflammatory response, amyloidosis and intraperitoneal hemorrhage, ultimately resulting in graft loss.

The next milestone in the field of islet transplantation would be development of a novel transplant site which is safe and has the efficacy to support the survival of transplanted islets. In this regard, the subcutaneous space is a particularly tempting site for islet transplantation owing to its safety profile and easy access. However, the paucity of nutrients and oxygen in the subcutaneous space inevitably tends to result in islet cell death, and many experimental trials of islets transplantation under the skin have failed to support engraftment and function of the transplanted islets.

The milieu in which islets are engrafted is critically important for their survival. Thus, there is a need to develop compositions and methods that enhance the survival and engraftment of islet cells in islet transplantation.

### SUMMARY OF THE INVENTION

This disclosure provides a unique tissue culture media (TCM) that when mixed with isolated islets has uniformly allowed successful engraftment and/or survival of human islets under the skin of immunodeficient diabetic mice. It has been confirmed that the mixture of this TCM with isolated islets when transplanted under the skin provide robust glucose control. Based on the success of this preclinical model, this TCM technology would be expected to provide significant improvement in the survival and engraftment of islet cells for islet transplantation.

In one aspect, this disclosure provides compositions to facilitate the survival and engraftment of cells (e.g., isolated islets) for transplantation. In one embodiment, the composition comprises collagen I, albumin, L-glutamine and NaHCO₃. In one embodiment, the collagen I is human collagen I and the albumin is human albumin.

In another aspect, this disclosure also provides methods of using the culture media disclosed herein. In one embodiment, provided herein are methods of using the media disclosed herein for transplanting β-cells of pancreatic islets into a site of a host. In one embodiment, the method comprises the steps of mixing a population of β-cells with the composition disclosed herein, and then transplanting the mixture of cells into a site of a host.

This disclosure also provides pharmaceutical compositions comprising collagen I, albumin, L-glutamine, NaHCO₃ and autologous β-cells of pancreatic islets of a subject. This disclosure also provides methods of treating Type I diabetes in a subject in need thereof, comprising administering a pharmaceutical composition comprising collagen I, albumin, L-glutamine, NaHCO₃ and autologous β-cells of pancreatic islets of a subject to a non-percutaneous trans-hepatic site in the subject. In an embodiment, the autologous β-cells of pancreatic islets are stem cell-derived β-cells.

This disclosure also provides pharmaceutical compositions comprising collagen I, albumin, L-glutamine, NaHCO₃ and autologous cells of a subject. In an embodiment, the autologous cells are endocrine and/or secretory cells, such as but not limited to, hepatocytes, parathyroid cells or adrenal gland cells. In certain embodiments, the autologous endocrine and/or secretory cells are stem cell-derived hepatocytes, parathyroid cells or adrenal gland cells.

This disclosure also provides methods of treating acute liver failure or hepatitis in a subject in need thereof, comprising administering a pharmaceutical composition comprising collagen I, albumin, L-glutamine, NaHCO₃ and autologous cells to a non-percutaneous trans-hepatic site in the subject, wherein the autologous cells are hepatocytes.

These and other aspects of the invention will be appreciated from the ensuing figure descriptions, detailed description of the invention, examples and figures. It should be understood, however, that the detailed description and specific examples while indicating certain embodiments of the invention are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1F show transplantation of murine, porcine and human islets admixed with a cell-free IVM (islet viability matrix), without any implanted device, thrive subcutaneously and maintain robust glycemic control. FIG. 1A shows a schematic of an experimental protocol for subcutaneous injection of murine, porcine or human islets admixed with or without cell-free IVM.

FIG. 1B shows the survival time of mice receiving subcutaneous injection of murine, porcine or human islets admixed with or without cell-free IVM. FIG. 1C shows blood glucose levels in diabetic immune-incompetent mice receiving human islets grafted subcutaneously with or without cell-free IVM. FIG. 1D shows how the IVM surrounds the islet clusters and becomes fully integrated with the cells. FIG. 1E shows grafting macaque islets with IVM underneath the skin rendered the animal normoglycemic. FIG. 1F shows that across different time points (post-transplant days 72 and 918), islet morphology and function was preserved.

FIGS. 2A-2I show the mechanism by which IVM imparts enhanced viability to the subcutaneously transplanted human islets is mediated by anti-apoptotic and pro-angiogenic signals. FIG. 2A shows Cryo-Electron Microscopy (CEM) image of human extracellular microvesicles isolated from recipient mouse plasma shows intact nanovesicles (40-100 nm in size). A representative graph shows nanoparticle tracking of isolated vesicles on the NanoSight 300 detector for concentration and size distribution (<105 nm). FIG. 2B shows circulating human TISEs isolated at 6 hr, 12 hr, and on PODs 1, 3 and 10 from the IVM⁺ and IVM⁻ cohorts were sequenced. Differential expression across the five time points analyzed using one-sided Mann-Whitney U, as summarized in the Volcano Plot (FIG. 2B). The x-axis is log₂ ratio of gene expression levels between the two cohorts; the y-axis is the Benjamin Hochberg-adjusted p-value based on −log₁₀. The colored dots represent the differentially expressed genes (blue=lower expression; red=higher expression in the IVM⁺ cohort) based on p<0.05 and two-fold expression difference. FIG. 2C shows the highest-expressing miRNAs in TISEs from IVM⁺ samples and their relative levels in both cohorts. FIG. 2D shows that a Gene Set Enrichment Analysis (GSEA) identified the major pathways (FDR≤0.05) that were cither upregulated or downregulated in the IVM⁺ group. FIG. 2E shows circulating human TISEs from IVM⁺ group showed higher expression of β-cell-specific proteins and anti-apoptotic markers as part of its intraexosomal cargo by Western blot analysis. TSG101 protein is shown as a canonical exosome marker. FIG. 2F shows SEMs of subcutaneously implanted islets (‘I’) demonstrate enhanced survival with IVM. Despite appearing healthy and granular (‘G’) on POD1, the islets implanted in subcutaneous tissue (‘SC’) without IVM show severe apoptotic blebbing (‘B’), vacuolization (‘V’), and loss of structural integrity, indicative of graft failure on POD7. In contrast, the Islet-IVM (‘M’) mixture maintains a healthier and granular phenotype without visibly presenting any morphological indications of apoptosis or necrosis. FIG. 2G shows based on human-into-mouse islet transplant model, immunohistochemical profiling of islets±IVM for markers of angiogenesis (VEGF), anti-apoptosis (Bcl-2, GLP-1) and endothelial cells (VWF) that highlights the increased intensity and stained area of these epitopes in the IVM⁺ group. The proportion and intensity of staining, quantified by QuPath using five automated regions of interest (ROIs) per sectioned image, is plotted below each section (* denotes p<0.05 and ** denotes p<5×10-5 based on the one-way Kruskal-Wallis H Test; arrow in the GLP-1 IVM-section denotes staining artifact). The particular significant p-values were—VEGF at POD 1 (p=0.009), Bcl-2 at POD1 (p=7×10-8) and POD10 (p=10-9), GLP-1 at POD1 (p=10-8) and POD 10 (p=4×10-9) and VWF at POD 10 (p=2×10-4). FIG. 2H shows blood glucose levels of diabetic mice transplanted with human islets±IVM (n=4 for all groups) showed that within 6 hours, animals in the IVM⁺ cohort are rendered normoglycemic. On POD 2, the grafts were excised to correlate gene expression with function. FIG. 2I shows levels of SLC2A2, INSULIN, PDX1 and VEGF in IVM⁻ (white bars; n=4) and IVM⁺ (black bars; n=4) islet groups were analyzed by RT-PCR. Relative mRNA levels were normalized to HPRT and expressed as mean±SEM. * denotes p<0.05 and ** denotes p<0.002.

FIGS. 3A-3H show results of syngeneic and xenogeneic islet transplantation experiments in the subcutaneous space, showing that excision of islet-bearing skin leads to recurrent diabetes. FIGS. 3A, 3C, 3E, and 3G show murine or porcine islet grafts were transplanted with IVM in immunoincompetent diabetic mice, following which non-fasting blood glucose level returned to physiological ranges (<200 mg/dl) and remained stable long term. Hyperglycemia promptly resumed upon removal of the grafts (indicated by downward arrows in FIGS. 3A, 3C, 3E, and 3G. Additionally, the presence of viable and functional transplanted islets from donor mice FIG. 3B and FIG. 3F and pigs FIGS. 3D and 3H in the subcutaneous space were established by histologic examination and staining for insulin (red) and glucagon (green).

FIGS. 4A-4B show scanning electron micrographs of human islets (‘I’) 7 days after implantation into subcutaneous tissue (‘SC’). FIG. 4A shows apoptotic and necrotic features accompany a progressive loss of structural integrity in the islet tissues without IVM, causing progressive loss of function and physical degradation. FIG. 4B shows islets implanted in IVM maintain a characteristically healthy morphology devoid of blebbing or intra-islet structural degradation; the IVM matrix (‘M’) surrounds islets and enhances their engraftment and survival.

FIGS. 5A-5C show the kinetics of glucose disposal when animal recipients bearing long-term subcutaneous islets underwent glucose tolerance testing. Long-term recipients (n=5 in each group) bearing IVM⁺ islets from mice (FIG. 5A), swine (FIG. 5B), or humans (FIG. 5C), demonstrated normal glucose disposal, akin to healthy non-diabetic mice.

FIGS. 6A-6B show measurements of human C-peptide levels in long-term recipients with or without IVM⁺ islets from mice, swine or humans, and expression of the primary glucose receptor, Glut2 (SLC2A2) and insulin in human islets cultured in the presence or absence of IVM. FIG. 6A shows human C-peptide levels measured in serum of recipients with subcutaneous human islets with and without IVM. FIG. 6B shows expression of primary glucose receptor, Glut2 (SLC2A2), or insulin in human islets cultured in the presence or absence of IVM for 0, 3 or 7 days.

FIGS. 7A-7C show results of Ki67 staining performed on human islets transplanted subcutaneously in immunoincompetent diabetic mice, with and without IVM. FIG. 7A shows Different cohorts of mice on PODs 1, 7 and 10 demonstrated enhanced Ki67 intensity in the IVM⁺ cohort. FIG. 7B shows grafts excised after 1 week were immunoassayed for insulin (green), BrdU (red) and counterstained for nuclear DNA with DAPI (blue). Yellow arrows point to cells with DNA replication as indicated by BrdU incorporation. FIG. 7C shows quantification of DNA replication rate in IVM⁺ and IVM⁻ cohorts (n=4) shows increased replication in the IVM⁺ group using Wilcoxon Rank-Sum Test.

FIGS. 8A-8B show morphological and immunohistochemical analysis of autologous cynomolgus monkey islets, implanted in the subcutaneous space that was performed at the time of euthanasia (animal ID #212077, POD 918). Abundant healthy islet cell clusters, exhibiting vivid expression of key markers such as Insulin, Glucagon, Bcl-2, GLP-1, Ki67, VEGF, VWF and Collagen were found. Due to IACUC regulations and ethical care guidelines for nonhuman primate research, subcutaneous autologous islet transplants without IVM as a control could not be performed in a cynomolgus monkey.

FIG. 9 shows that β-cell morphology and endocrine function is maintained in retroperitoneal islet transplantation admixed with IVM.

FIG. 10 shows a schematic depicting GLPIR signaling pathway.

FIGS. 11A-11D show pancreatic islets transplanted in the subcutaneous space with IVM promote optimal glucose homeostasis in immune-incompetent diabetic hosts. FIG. 11A shows murine, porcine or human islets were admixed with or without IVM and grafted subcutaneously. Individual islet graft survival across different animal models is summarized. Islets transplanted without IVM uniformly resulted in primary non-function. In all cases, the number of days given for the IVM⁺ group represents excision of the islet bearing skin at the times of elective retrieval and not due to graft destabilization. SCID, severe combined immune deficiency. FIG. 11B shows metabolic homeostasis, as evaluated by glucose measurements in B6/SCID animals transplanted with human islets±IVM, showed that IVM⁺ islets consistently rendered the recipients normoglycemic. FIG. 11C shows human C-peptide levels were measured in the serum of these recipients and are shown in the violin plot. Each dot represents C-peptide measured from an individual recipient mouse. The difference in C-peptide levels was statistically significant (***P<10⁻⁵ based on the one-sided Mann-Whitney U Test). FIG. 11D shows as a representative example, in B6/SCID mice, at POD7 in the IVM⁻ cohort and POD49 in the IVM⁺ cohort, an excisional biopsy was performed, showcasing fragmented insulin⁺ cells in the former group, in contrast to preserved islet architecture and integrated collagen in the latter. Images show the results from H&E staining and IHC (red, insulin; green, glucagon).

FIGS. 12A-12C show pancreatic islets transplanted in the subcutaneous space with IVM promote optimal glucose homeostasis in immune-competent recipients. FIG. 12A shows immunotherapy regimen targeting T- and B-cell compartments to promote islet graft survival in immune-competent diabetic hosts. mAb, monoclonal antibody. FIG. 12B shows survival data for islet allografts and xenografts transplanted subcutaneously in B6 mice. The numbers of days given for the IVM⁺ group represent excision of islet-bearing skin at the times of elective retrieval and not due to destabilization of the grafts, unless the time is indicated by superscript ϕ. FIG. 12C show H&E and IHC (red, insulin; green, glucagon; purple, CD8) of islet-bearing skin in long-term normoglycemic recipients of IVM⁺ islet grafts showing abundant clusters of healthy α and β cells.

FIGS. 13A-13B show the islet-IVM mixture of pancreatic islets transplanted in the retroperitoneal space renders the recipients normoglycemic. FIG. 13A demonstrates individual islet graft survival±IVM in the retroperitoneal space of immune-incompetent diabetic mice for experiments utilizing murine, porcine or human islets. In all cases, the numbers of days given for the IVM⁺ group represent excision of the islet-bearing skin at the times of elective retrieval and not due to destabilization of the grafts. Islets transplanted without IVM uniformly resulted in primary non-function. Green, representative days for which the histology is shown. FIG. 13B shows β-cell morphology and endocrine function is maintained in retroperitoneal islet. transplantation admixed with IVM. The top panels show H&E stains from murine, porcine and human grafts, displaying abundant viable cell clusters in the islets during long-term follow-up post-transplantation. In conjunction, the bottom panels substantiate the presence, via IHC of insulin (red) and glucagon (green), correlating with the functional status of the grafted islets.

FIGS. 14A-14C show IVM imparts enhanced viability to subcutaneously transplanted human islets, as reflected in anti-apoptotic and pro-angiogenic signals. FIG. 14A shows blood glucose levels of diabetic B6 SCID mice transplanted with 500 human islets±IVM (n=4 animals in each group) showed that within 6 hours, animals in the IVM⁺ cohort were rendered normoglycemic. Mean glycemic values for each experimental group are plotted, and error bars represent s.d. **P=4×10⁻⁷, 3×10⁻⁸ and 8×10⁻⁶ (at 6, 24 and 48 hours, respectively) between the IVM⁺ and IVM⁻ groups by using one-sided Mann-Whitney U-test. At time 0 h, age-matched mice with similar baseline glucose homeostasis were transplanted with human islets±IVM. On POD 2, the grafts were excised to correlate gene expression with function. FIG. 14B shows levels of SLC2A2 (P=0.002), INSULIN (P=0.01), PDX1 (P=0.003) and VEGF (P=0.43) in IVM⁻ (white bars; n=4 micc) and IVM⁺ (black bars; n=4 mice) islet groups were analyzed by RT-PCR; each dot represents an individual data point. Relative mRNA levels are normalized to hypoxanthine phosphoribosyltransferase and are expressed as mean±s.e.m. (*P<0.05 and **P<0.005 based on one-sided Student's t-test). FIG. 14C shows results of a male cynomolgus monkey (M. fascicularis) that underwent ˜90% pancreatectomy, and 5 h later, underwent subcutaneous implantation of isolated islets with IVM. During this interval, glucose levels were elevated (>200 mg/dL) but remained in the normal range after auto-transplantation. On POD 72 (left), a partial excisional biopsy was performed, demonstrating abundant clusters of islets with robust expression of insulin and glucagon (red and green, respectively, on IHC). Longitudinal monitoring of blood glucose in this monkey revealed optimal glucose homeostasis until POD 820 when we observed progressive evidence of hyperglycemia mandating administration of exogenous insulin. Because this monkey became overtly diabetic, per IACUC it was subjected to euthanasia on POD 918 (right). Importantly, histological examination of the skin at the site of islet implantation demonstrated the persistence of well-granulated islet endocrine clusters in the subcutaneous space with no evidence of fibrosis and inflammatory cell infiltration.

FIGS. 15A-15B shows for cynomolgus monkey ID #210069, the animal's blood glucose just prior to pancreatectomy was 72 mg/dl; blood glucose monitoring post-transplantation demonstrated persistent hyperglycemia in the animal, which required management by exogenous insulin therapy. FIG. 15A shows that failure to achieve normoglycemia in this monkey can be attributed partly to the suboptimal islet yield and transplantation of a relatively low mass of islets (11,827 IEQs/kg body weight), as well as the infusion of streptozotocin which likely led to the destruction of both native remnant islets as well as subcutaneously transplanted islets. FIG. 15B shows H&E and IHC staining of the islet bearing skin that were performed on POD 46 and POD 250. In view of the persistent state of insulin dependency and per the recommendation of the IACUC veterinarian, the monkey was subjected to euthanasia on POD 250. During this course, an excisional biopsy of the islet bearing skin was performed on POD 46 and at the time of euthanasia (POD 250). Both histologic assessments revealed abundant well granulated islet β-cells as well as glucagon-positive a-cells.

### DETAILED DESCRIPTION OF THE INVENTION

The present invention may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one.

Throughout this application, various embodiments of this disclosure may be presented in a range format. It should be understood that a description in range format is merely for convenience and brevity and should not be construed as an inflexible limitation on the scope of the invention. Accordingly, description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of +10% from the indicated number or range of numbers. In another embodiment, the term “about” refers to a deviance of +5% from the indicated number or range of numbers.

As used herein, the terms “component,” “composition,” “composition of compounds,” “compound,” “drug.” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject.” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates (particularly higher primates), sheep, dog, rodent, (e.g., mouse or rat), guinea pig, goat, pig, cat, rabbits, cows, horses and non-mammals such as reptiles, amphibians, chickens, and turkeys.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

As discussed above, the intrahepatic milieu is inhospitable to intraportal islet allografts, limiting their applicability to ameliorate Type 1 Diabetes (T1D). Although the subcutaneous space represents an alternate, safe and easily accessible site for pancreatic islet transplantation, lack of neovascularization and the resulting hypoxic cell death have largely limited the longevity of graft survival and function, and pose a barrier to widespread clinical adoption of islet transplantation. A crucial step to ensure widespread adoption and safety of clinical islet transplantation is islet implantation at an easily accessible site. This disclosure provides an Islet Viability Matrix (IVM) that can uniformly promote and maintain graft survival and function in the subcutaneous space, curing T1D through a previously unknown anti-apoptotic mechanism. The IVM described and provided herein is in one embodiment a mixture of human collagen I, L-glutamine, fetal bovine serum, sodium bicarbonate and medium 199, which when admixed with murine, porcine or human islets, promotes uniform islet survival subcutaneously. Described herein is the successful subcutaneous transplantation of pancreatic islets admixed with a device-free IVM, resulting in long-term euglycemia in diverse immune-competent and immuno-incompetent animal models.

Data presented herein indicate that subcutaneous pancreatic islet transplantation with IVM represents a novel approach and a viable alternative to the current standard for clinical islet transplantation in T1D. This disclosure provides a device-free system, involving a single injection of an islet-IVM mixture, that does not result in major avascular, hypoxic or fibrotic reactions in either the subcutaneous or retroperitoneal spaces. The protective effect of this IVM mixture is mediated, at least in part, by upregulation of anti-apoptotic signaling pathways. By preserving normal insulin and glucagon production, islets bedaubed with IVM maintain basal glycemia across mouse species as well as in a nonhuman primate model. Establishment of this method augments the utility of pancreatic islet transplantation, as well as related cellular therapies in tissue engineering and reparative medicine.

Many research efforts have focused on developing devices that could be seeded with whole islets (or stem cell-derived β-cell lines) with the intent to implant these “loaded devices” into a host. A distinct advantage of the IVM disclosed herein is that no device is needed. Employing a device in itself presents a big biologic challenge because host responses would be induced against the material or fabric of the foreign device, thereby leading to fibrosis and loss of islet viability.

In one aspect, the compositions provided herein facilitate the survival and engraftment of cells for transplantation, the composition comprises 10×M199, collagen I, albumin, L-glutamine and NaHCO₃. In one embodiment, the cells are human cells. In one embodiment, the cells are β-cells of pancreatic islets. In one embodiment, the collagen I is human collagen I. In one embodiment, the albumin is human albumin. In one embodiment, fetal bovine serum is used. In one embodiment, the L-glutamine:NaHCO₃ ratio is about 1:4. In one embodiment, the L-glutamine:albumin ratio is about 1:18. In one embodiment, the L-glutamine:collagen I ratio is about 1:160. In one embodiment, the L-glutamine: 10×M199 ratio is about 1:18.

In one embodiment, the composition comprises about 60% to 94% of collagen I. In another embodiment, the composition comprises about 4% to 15% of albumin. In one embodiment, the composition comprises about 0.4% to 4% of L-glutamine. In one embodiment, the composition comprises about 1% to 5% of NaHCO₃.

In another aspect, provided herein are methods of transplanting cells such as β-cells of pancreatic islets into a host site, comprising the steps of: mixing a population of β-cells with a media composition disclosed herein, and then transplanting this mixture of cells into the host site. The host can be a human or a non-human animal. In one embodiment, the β-cells are human, murine, or porcine β-cells. In another embodiment, the cells are alpha cells, delta cells or PP cells in the pancreas. In an embodiment, the endocrine cells are stem cell-derived endocrine cells. In an embodiment, the transplant into the host site is a xenotransplant. In certain embodiments, the host is a human and the β-cells are non-human β-cells, wherein the non-human β-cells are murine β-cells or porcine β-cells. In another embodiment, the host is a non-human animal and the β-cells are human β-cells. In one embodiment, the cells are transplanted into a subcutaneous space or retroperitoneal space. In another embodiment, the cells are transplanted into an omentum or an abdominal cavity.

In another aspect, provided herein are pharmaceutical compositions comprising collagen I, albumin, L-glutamine, NaHCO₃ and autologous β-cells of pancreatic islets of a subject. In an embodiment, the collagen I is human collagen I. In one embodiment, the albumin is human albumin. In some embodiments, the pharmaceutical composition further comprises fetal bovine serum. In some embodiments, the pharmaceutical composition comprises about 60% to 94% of collagen I. In an embodiment, the pharmaceutical composition comprises about 4% to 15% of albumin. In one embodiment, the composition comprises about 0.4% to 4% of L-glutamine. In various embodiments, the composition comprises about 1% to 5% of NaHCO₃.

In another aspect, provided herein are methods of treating Type I diabetes in a subject in need thereof, the method comprising: administering a pharmaceutical composition comprising collagen I, albumin, L-glutamine, NaHCO₃ and autologous β-cells of pancreatic islets of a subject to a non-percutaneous trans-hepatic site of the subject. In an embodiment, the non-percutaneous trans-hepatic site is a subcutaneous space or retroperitoneal space. In another embodiment, the cells are transplanted into an omentum or an abdominal cavity. In some embodiments, the subject is a human or an animal. In an embodiment, the collagen I is human collagen I. In one embodiment, the albumin is human albumin. In some embodiments, the pharmaceutical composition further comprises fetal bovine serum. In certain embodiments, the pharmaceutical composition comprises about 60% to 94% of collagen I. In one embodiment, the pharmaceutical composition comprises about 4% to 15% of albumin. In one embodiment, the composition comprises about 0.4% to 4% of L-glutamine. In some embodiments, the composition comprises about 1% to 5% of NaHCO₃.

Also provided herein are pharmaceutical compositions comprising collagen I, albumin, L-glutamine, NaHCO₃ and autologous cells of a subject. In some embodiments, the autologous cells are endocrine and/or secretory cells, such as but not limited to, hepatocytes, parathyroid cells or adrenal gland cells. In certain embodiments, the endocrine cells are alpha cells. In an embodiment, the endocrine cells are stem cell-derived endocrine cells. In certain embodiments, the autologous endocrine and/or secretory cells are stem cell-derived hepatocytes, parathyroid cells or adrenal gland cells. In an embodiment, the collagen I is human collagen I. In some embodiments, the albumin is human albumin. In certain embodiments, the pharmaceutical composition further comprises fetal bovine serum. In an embodiment, the pharmaceutical composition comprises about 4% to 15% of albumin. In one embodiment, the composition comprises about 0.4% to 4% of L-glutamine. In various embodiments, the composition comprises about 1% to 5% of NaHCO₃.

Also provided herein are methods of treating acute liver failure or hepatitis in a subject in need thereof, the method comprising: administering a pharmaceutical composition comprising collagen I, albumin, L-glutamine, NaHCO₃ and autologous cells to a non-percutaneous trans-hepatic site of the subject, wherein the autologous cells are hepatocytes. In an embodiment, the autologous hepatocytes are stem cell-derived hepatocytes. In one embodiment, the non-percutaneous trans-hepatic site is a subcutaneous space or retroperitoneal space. In another embodiment, the cells are transplanted into an omentum or an abdominal cavity. In some embodiments, the subject is a human or an animal. In one embodiment, the collagen I is human collagen I. In one embodiment, the albumin is human albumin. In some embodiments, the pharmaceutical composition further comprises fetal bovine serum. In certain embodiments, the pharmaceutical composition comprises about 60% to 94% of collagen I. In an embodiment, the pharmaceutical composition comprises about 4% to 15% of albumin. In one embodiment, the composition comprises about 0.4% to 4% of L-glutamine. In some embodiments, the composition comprises about 1% to 5% of NaHCO₃.

In this description, each of the steps of the invention and variations thereof are described. This description is not intended to be limiting and changes in the components, sequence of steps, and other variations would be understood to be within the scope of the present invention.

In one aspect, provided herein are pharmaceutical compositions comprising collagen I, albumin, L-glutamine, NaHCO₃ and autologous β cells of pancreatic islets of a subject. In one embodiment, the pharmaceutical composition comprises about 60% to 94% of collagen I. In one embodiment, the pharmaceutical composition comprises about 4% to 15% of albumin. In one embodiment, the pharmaceutical composition comprises about 0.4% to 4% of L-glutamine. In one embodiment, the pharmaceutical composition comprises about 1% to 5% of NaHCO₃. In one embodiment, the composition further comprises fetal bovine serum.

Various embodiments and aspects of the present invention as delineated hereinabove and as claimed in the claims section below find experimental support in the following examples.

The following examples are presented in order to illustrate certain embodiments of the invention more fully. The examples should in no way be construed, however, as limiting the broad scope of the invention.

### Example 1

### Islet Viability Matrix (IVM) for the Survival and Engraftment of Human Pancreatic Islets

### Materials and Methods

### Affinity Antibody-Coupled Bead Purification of Tissue Specific EVs

MHC specific antibody was covalently conjugated to N-hydroxysuccinamide magnetic beads (Pierce) per manufacturer's protocol. Fifty to 100 μg protein equivalent of EVs were incubated with antibody beads overnight at 4° C. The bead bound and unbound EV fractions were separated per manufacturer's protocol. EVs bound to beads were eluted using tris glycine and utilized for downstream analysis.

### Animals and Induction of Diabetes

Male C57BL/6 (B6) and B6.CB17-Prkdcscid (B6 SCID) mice aged 8-12 weeks, used as islet donors and recipients, were obtained from the Jackson Laboratory, Bar Harbor, ME. Female BALB/c and C3H mice aged 8-12 weeks were used as allogeneic islet donors. Recipients were rendered diabetic by a single intraperitoneal injection of streptozotocin (SICOR Pharmaceuticals, Inc. Irvine, CA) at a dose of 250 mg/kg. At 5 days after streptozotocin administration, animals with two consecutive (daily) non-fasting blood glucose levels>350 mg/dL (Accu-Chek Glucometer, Roche Diagnostics, Indianapolis, IN) were used as islet recipients. The protocol was approved by the University of Pennsylvania's Institutional Animal Care and Use Committee, and in accordance with the National Institutes of Health's Guide for the Care and Use of Laboratory Animals.

### Assessment of Islet Graft Function

Blood Glucose levels were monitored twice weekly after transplantation and recipients with non-fasting glucose concentrations<200 mg/dL were considered to have achieved normoglycemia. When two consecutive daily non-fasting glucose levels were >300 mg/dL after a period of primary graft function, islet grafts were considered to have failed. An intraperitoneal glucose tolerance test was performed on the animals with long-term normoglycemia (>100 days). In select recipients, the site bearing the islet grafts (skin or retroperitoneum) were excised and the tissues were analyzed for immunohistochemical confirmation of isolated islets.

### Biochemical Analysis of Exosome Content

For total RNA (including microRNA and mRNA) purification, RNA was extracted from EVs using Trizol, followed by RNeasy mini kit, per manufacturer's protocol (Qiagen, Germany). For total protein isolation, EV pellet was lysed in 1×RIPA buffer with 1× concentration of protease inhibitor cocktail (Sigma-Aldrich Co., MO). For Western Blot analysis, EV and cell lysate total proteins were isolated and separated on polyacrylamide gels and transferred on polyvinylidene difluoride membrane (Life Technologies, NY). The blot was blocked, incubated with desired antibody at concentration per manufacturer's protocol. Horseradish peroxidase coupled secondary antibody (Santa Cruz Biotechnologies Inc.) was added per manufacturer's protocol and detected through chemiluminescence using Image quant LAS 400 Phospho-Imager (GE Health, USA).

TISE and cell total RNA representing the transplanted human xenoislet mass was analyzed using the Agilent 2100 Bioanalyzer and Nanodrop spectrophotometry at the University of Pennsylvania Molecular Profiling Facility. All protocols were performed according to the Qiagen QIAseq miRNA sample prep kit (catalogue #331502).

### Extracellular Vesicle (EV) Isolation

EVs were isolated by high exclusion limit agarose-based gel chromatography along with ultracentrifugation. Briefly, 500 μL to 1 mL plasma was obtained from the islet recipient after centrifugation of the blood sample at 500 g for 10 minutes. To eliminate cells and debris, the solution was filtered through a 0.22 μm filter and then passed through a Sepharose 2B column. The eluent was collected in 1 mL fractions. The EV fraction was pooled after monitoring absorbance at 280 nm. The pooled fraction underwent ultracentrifuge at 110,000 g for 2 hours at 4° C., and the pelleted EV fraction was resuspended in PBS for downstream analysis.

Anti-HLA-A2 antibodies (Santa Cruz Biotechnologies, TX) were utilized for NanoSight fluorescent staining and analysis of human islet EVs purified from recipient mouse plasma. Antibodies to human FXYD2 (Abnova), insulin, glucagon, somatostatin, CD3, CD4, CD8, CD56, CD19, CD56, TSG101, aquaporin 2, podocalyxin-1, and to mouse MHC I were purchased from Santa Cruz Biotechnologies. Secondary antibodies and isotype controls (anti-goat, anti-rabbit, anti-mouse, goat IgG, rabbit IgG, and mouse IgG) were also purchased from Santa Cruz Biotechnologies. Anti-goat, anti-rabbit, and anti-mouse conjugated quantum dot (605 nm) were purchased from Life Technologies (Grand Island, NY) and utilized per manufacturer's protocol for NanoSight fluorescent analysis.

### EV Sequencing Analysis

The read layouts obtained from EV sequencing were processed. First, the sequencing adapter was trimmed, and then reads were taken that had exact matches to the inner fixed region and extracted the flanking sequences as RNA and UMI respectively. Alignments were done with bowtie using the flags -q -k 4 --best --norc. Duplicate UMIs were reduced by transcript, by removing duplicates that align to the same read. The frequency of similar UMIs produced by sequencing errors was small, and normalization was performed by converting the read counts to CPM. For the miRNA analysis, exosome sequences were aligned to miRbase release 21. All of the target libraries—miRNA, tRNA, and RefSeq—had duplicate entries, therefore the best alignments were taken and allowed reads to map to multiple places.

### Immunohistochemistry

The proportion and intensity of the stained epitopes were evaluated using QuPath v0.1.2.

### Islet Isolation And Transplantation

Mouse pancreatic islet isolation was performed by collagenase P (Roche Diagnostics, Indianapolis, IN) digestion and density gradient separation. Human isolated pancreatic islets were procured from deceased organ donors through an Integrated Islet Distribution Program with consent from the regional Organ Procurement Organization (Gift of Life Donor Program). Islets were incubated in CMRL 1066 medium (Mediatech, Manassas, VA) containing 5.5 mM d-glucose, 0.5% human albumin (Talecris Biotherapeutics, Research Triangle Park, NC), 10 U/mL Heparin (Sagent Pharmaceuticals, Schaumberg, IL), 100 μg/mL penicillin/streptomycin, and 2 mM L-glutamine. Porcine islets were obtained from Dr. Bernhard Herring at the University of Minnesota.

The recipient mice were anesthetized by an intraperitoneal injection of Ketamine HCl (50 mg/kg body weight; Abbott Laboratories, North Chicago, IL). In the subcutaneous (SC) transplantation model, a small skin incision was established over the lower abdomen to create a SC pocket in which islets were injected immediately after isolation in either a suspension of 250 μL of RPMI-1640 (“islets alone”) or admixed in tissue culture enriched of Collagen I (Organogenesis, Canton, MA), human albumin, L-glutamine and NaHCO₃ (“IVM.” islet viability matrix). In the retroperitoneal (RP) transplantation model, the mouse abdominal cavity was opened, and islets were injected underneath the peritoneal layer in the right posterior retroperitoneal space. Islet transplantation was performed at the University of Pennsylvania per procedural protocols (CIT07 and CIT06). Islet viability, quantity, and function were analyzed by the Islet Core Facility per institution approved protocols.

### NanoSight EV Analysis

Purified EVs were analyzed on the NanoSight NS300 (405 nm laser diode) on the light scatter mode for EV quantification and scatter distribution according to manufacturer's protocols (Malvern instruments Inc., MA, USA). Before each experimental run, the machine was calibrated for nanoparticle size and quantity using standardized nanoparticle and dilutions provided by the manufacturer. Surface marker detection on EVs was performed using the fluorescence mode on the NanoSight NS300. Secondary antibodies conjugated to quantum dots with emission at 605 nm were utilized for fluorescence detection of primary antibodies binding against specific surface proteins. Each experimental run was performed in duplicates, and an appropriate IgG isotype control fluorescence was performed to assess background.

### Nonhuman Primate

Male and a female cynomolgus monkeys (M. Fascicularis) weighing 3.0-4.0 kg were used for autologous islet transplantation. The monkeys were obtained from the vendors Spring Scientific (Perkasie, PA). Prior to use for transplantation, serological testing was performed on each animal to ensure that they were not infected with Herpes B. Schmidt-Ruppin strain of the Rous sarcoma virus, simian T-cell lymphotropic virus, or simian immunodeficiency virus. All animals had continuous water supply and were fed with regular primate diet supplemented with fresh fruits twice daily. All animal care and handling were performed in accordance with the guidelines established by the Department of Health and Human Services' guide for care and use of primates, as well as the IACUC guidelines.

Pancreatectomy. Monkeys were sedated using ketamine (15 mg/kg) and atropine (0.05 mg/kg), prepared for operation, and intubated. Anesthesia was initiated with midazolam (1 mg/kg) and maintained with isoflurane and oxygen. Haircoat was clipped closely from the nipples to mid-thigh and laterally to the end of the vertebral processes and skin, followed by scrubbing with a preliminary chlorhexidine solution. Animals were placed in a supine position, and the surgical field given a second chlorhexidine scrub. The abdomen was opened using a midline laparotomy. The spleen and greater omentum were reflected to expose the entire pancreas. The right and left pancreatic limbs were mobilized, preserving pancreatic vasculature, and the pancreas then carefully dissected from the duodenal serosa. The common pancreatic duct was identified at the duodenum, ligated, and transversely incised distal to the ligation. Through this incision, pancreatic duct was cannulated with an angiocatheter, and secured with ligature. The pancreas is then excised, placed in cold UW's solution and transported to the islet isolation laboratory. Finally, the incisions are closed layer by layer.

Subcutaneous Islet Transplantation. Under general anesthesia, the animals were placed in a supine position, and the transplant field prepared. The solution containing isolated islets was slowly infused in the subcutaneous area. The needle point was then swabbed with an antiseptic. Finally, the animal was recovered in an incubator and extubated as soon as possible.

Islet graft function. The blood glucose levels were monitored twice daily for the first month then twice weekly after transplantation and recipients with non-fasting glucose concentrations<200 mg/dL were considered to have achieved normoglycemia. When two consecutive daily non-fasting glucose levels were >300 mg/dl after a period of primary graft function, islet grafts were considered to have failed. Islet graft biopsies were performed under general anesthesia. Tissue samples (1-2 cm²) were processed for standard hematoxylin-eosin (H&E) and immunohistochemistry (IHC) staining.

### Replication Assay

In vivo bromodeoxyuridine (BrdU) labeling was achieved by diluting drinking water with (1 mg/mL) BrdU (Sigma) for 3 days. BrdU staining was performed using the Zymed BrdU Staining Kit according to the manufacturer's instructions. BrdU-positive nuclei were counted blinded from at least 20 islets per mouse from four to six mice per group. The protocol was approved by the Institutional Animal Care and Use Committee of the University of Pennsylvania.

### RNA Isolation and Real-Time PCR Analysis

Islets from overnight fasted 18-wk-old mice were isolated using the standard collagenase procedure as previously described. Total RNA from islets was isolated in Trizol (Invitrogen) according to the manufacturer's instructions. Islet RNA was reverse-transcribed using 1 μg of Oligo(dT) primer, SuperScript II Reverse Transcriptase, and accompanying reagents (Invitrogen). PCR reaction mixes were assembled using the Brilliant SYBR Green QPCR Master Mix (Stratagene). Reactions were performed using the SYBR Green (with Dissociation Curve) program on the Mx4000 Multiplex Quantitative PCR System (Stratagene). All reactions were performed in triplicate with reference dye normalization, and median CT values were used for analysis. Islet purity was assessed and was determined to be >90% endocrine tissue.

For exosome analysis, total RNA (25 to 50 ng) from islet cells and EVs were reverse-transcribed with the SuperScript III one-step RT-PCR system (Life Technologies) for gene expression validation. The primers used were:

human insulin
(forward)
(SEQ ID NO: 1)
5′-CCTTGTGAACCAACACCTG-3′,
(reverse)
(SEQ ID NO: 2)
5′-GTAGAAGAAGCCTCGTTCCC-3′ (80 bp);
human glucagon
(forward)
(SEQ ID NO: 3)
5′-CCCAAGATTTTGTGCAGTGGTT-3′,
(reverse)
(SEQ ID NO: 4)
5′-CAGCATGTCTCTCAAATTCATCGTG-3′ (80 bp);
human somatostatin
(forward)
(SEQ ID NO: 5)
5′-GATGCCCTGGAACCTGAAGA-3′,
(reverse)
(SEQ ID NO: 6)
5′-CCGGGTTTGAGTTAGCAGATCT-3′ (82 bp);
human FXYD2γa
(forward)
(SEQ ID NO: 7)
5′-ACTGGGTTGTCGATGGACGGT-3′,
(reverse)
(SEQ ID NO: 8)
5′-CGGCTCATCTTCATTGATTTG-3′ (188 bp);
human FXYD2γb
(forward)
(SEQ ID NO: 9)
5′-GACAGGTGGTACCTG-3′,
(reverse)
(SEQ ID NO: 10)
5′-CGGCTCATCTTCATTGATTTG-3′ (188 bp);
and
human β-actin primers are
(forward)
(SEQ ID NO: 11)
5′-CTGTACGCCAACACAGTGCT-3′,
(reverse)
(SEQ ID NO: 12)
5′-GCTCAGGAGGAGCAATGATC-3′ (127 bp).

### Scanning Electron Microscopy (SEM)

Paraffin-embedded samples of skin and subcutaneous tissues were sectioned longitudinally until the implanted islets were visually observed, thereby facing off the block to expose the site of engraftment. Subsequently, the tissue was deparaffinized in xylene, rehydrated through an ethanol series of 100%, 95%, 90%, 80%, 70%, 0% to DPBS. The tissues were then fixed overnight at 4° C. with a solution of 2.5% glutaraldehyde and 1.25% formaldehyde in 0.1 M sodium cacodylate buffer. The tissues were subsequently washed three times with DPBS, post-fixed for one hour with 1% osmium tetroxide and 1.5% potassium ferrocyanide in water, then washed three times with PBS and dehydrated in an ethanol series (50%, 70%, 80%, 90%, 95%×2, 100%×3, 5 min. each). The samples were then dried at the CO2 critical point, plasma sputtered with 80:20 gold:palladium alloy, and finally imaged with an FEI Quanta 250 Scanning Electron Microscope operating at high chamber vacuum with 5 kV beam energy.

### Statistical Analysis

Data are expressed as mean±standard deviation. The significance of differences between two independent groups was calculated by the Wilcoxon Rank Sum/Mann-Whitney U Test, unless otherwise specified. Differences were considered significant at P<0.05. The gene-set enrichment analysis was performed using the WEB-based Gene SeT Analysis Toolkit.

### Results

### Murine, Porcine and Human Islets Transplantation

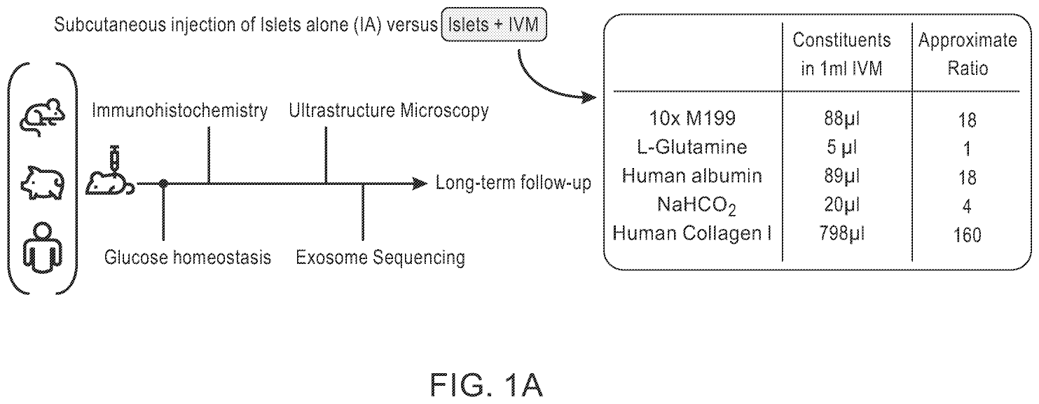

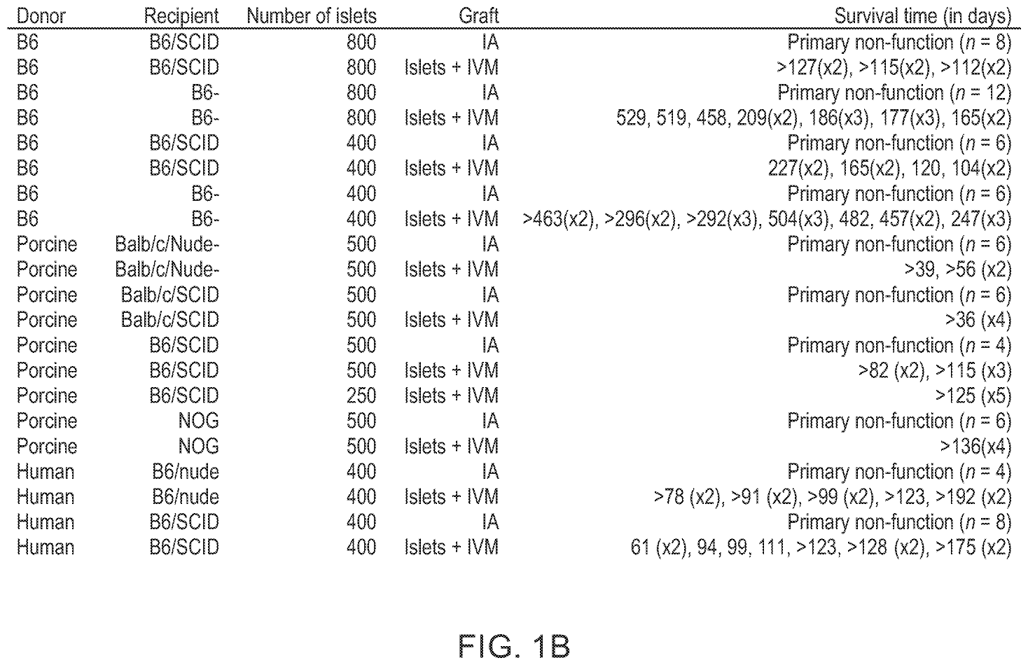

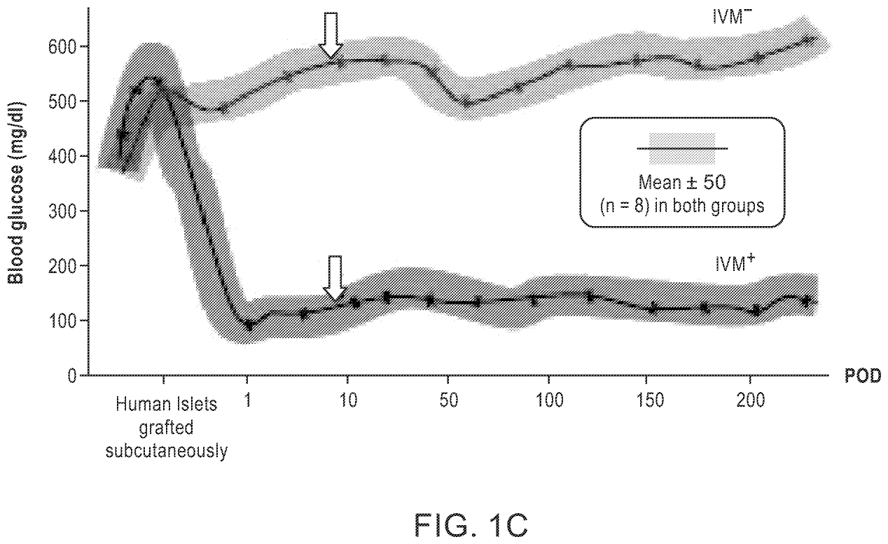

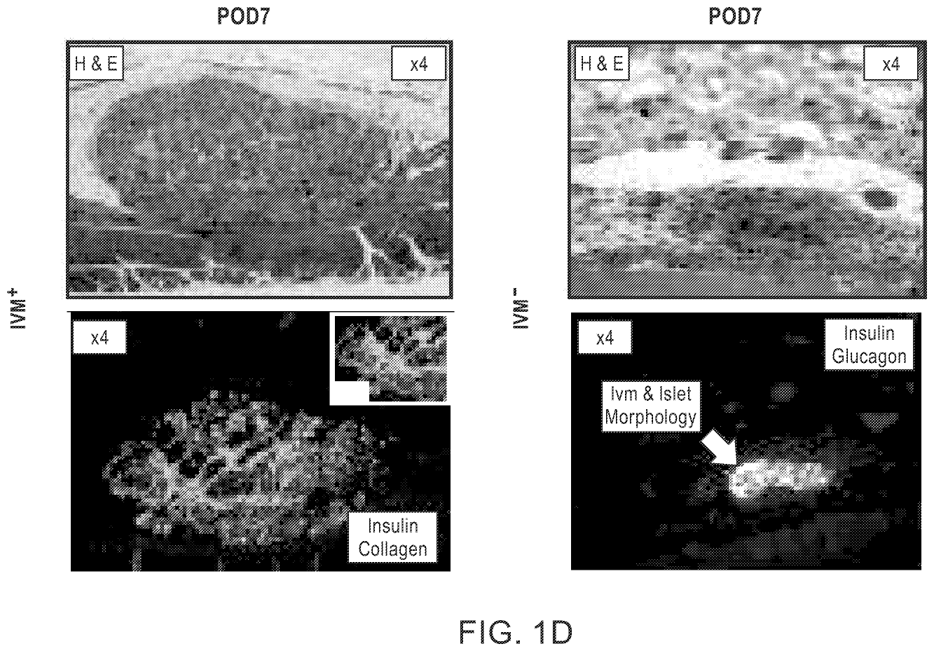

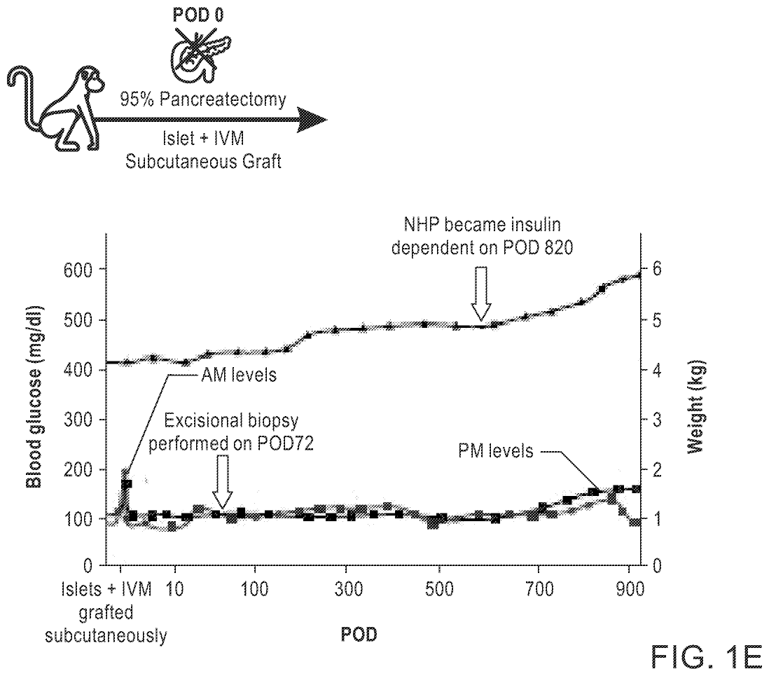

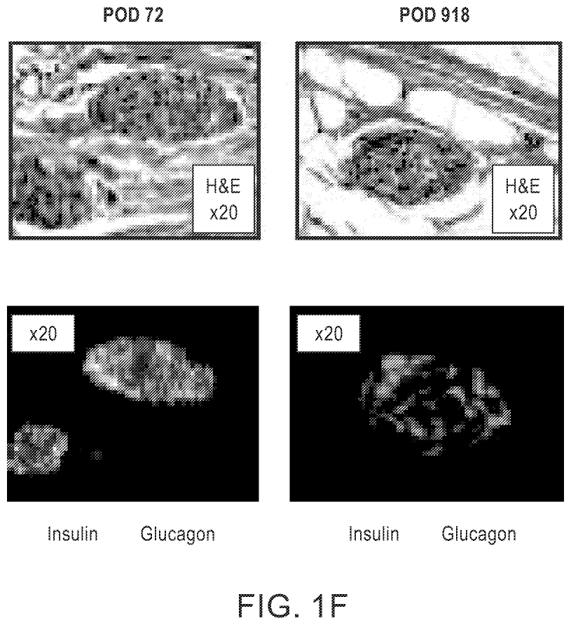

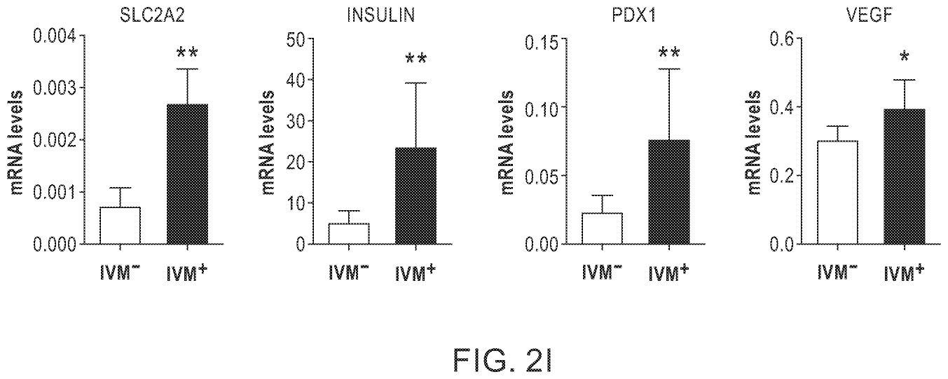

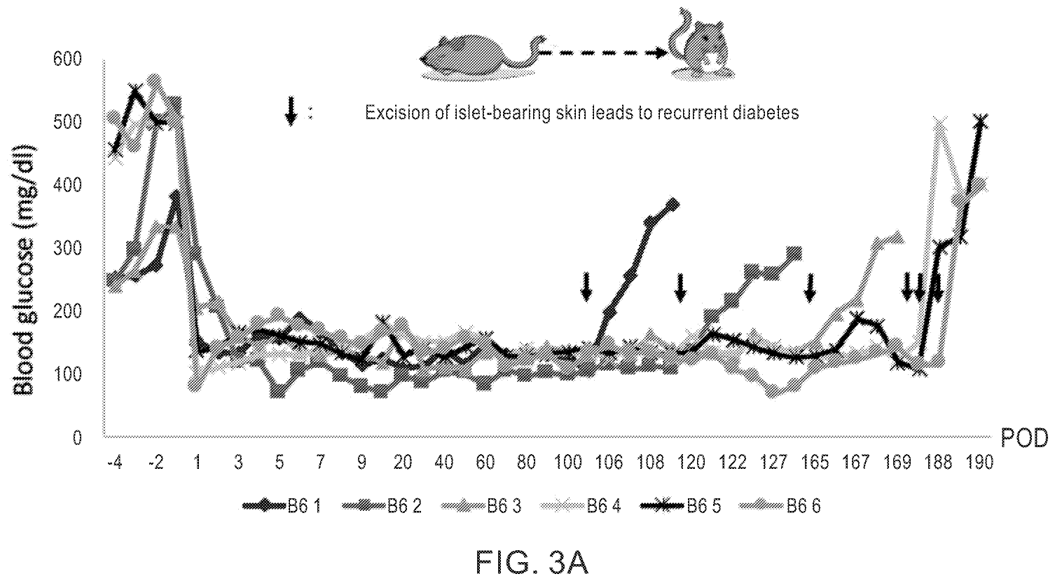

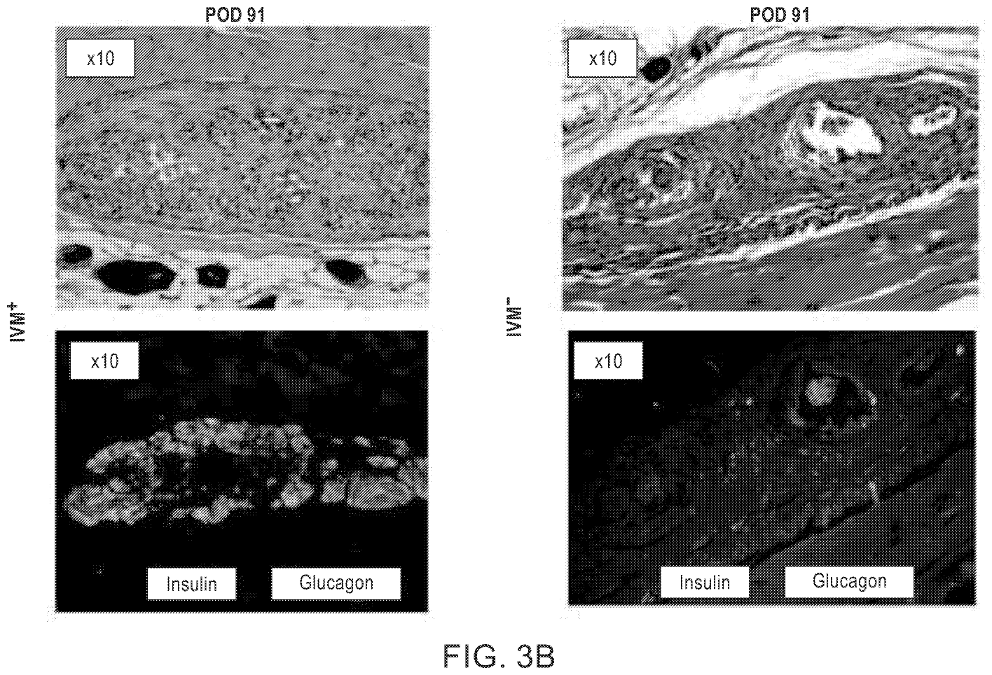

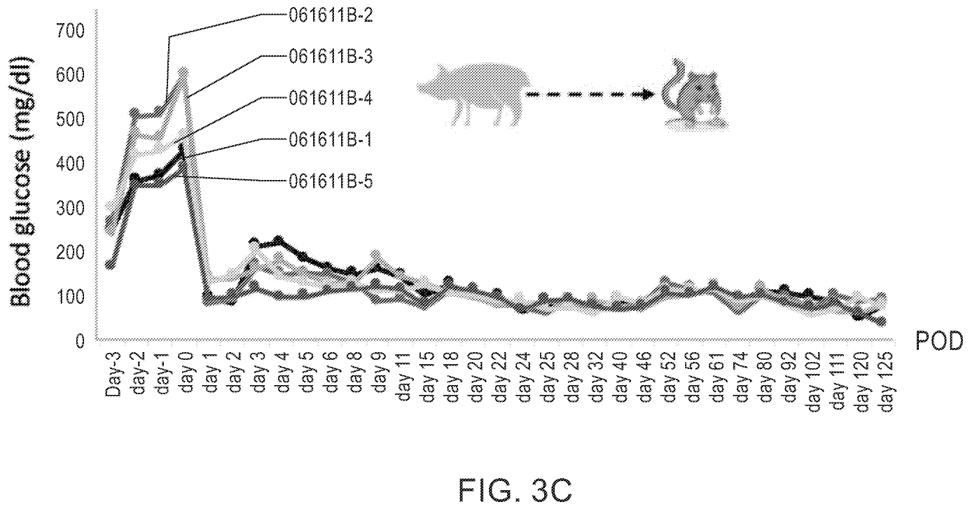

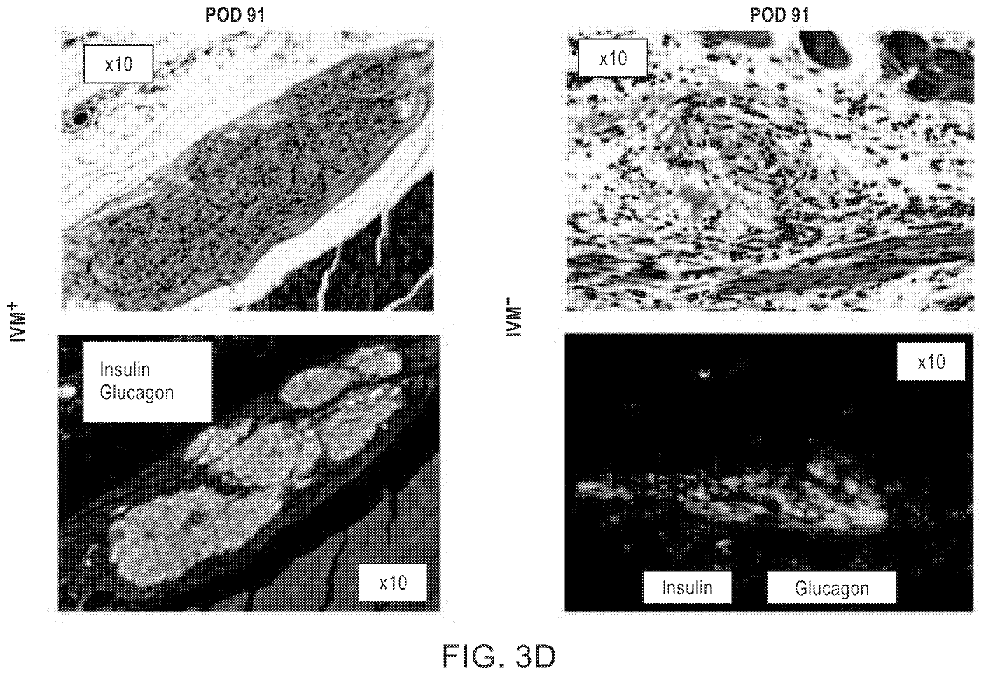

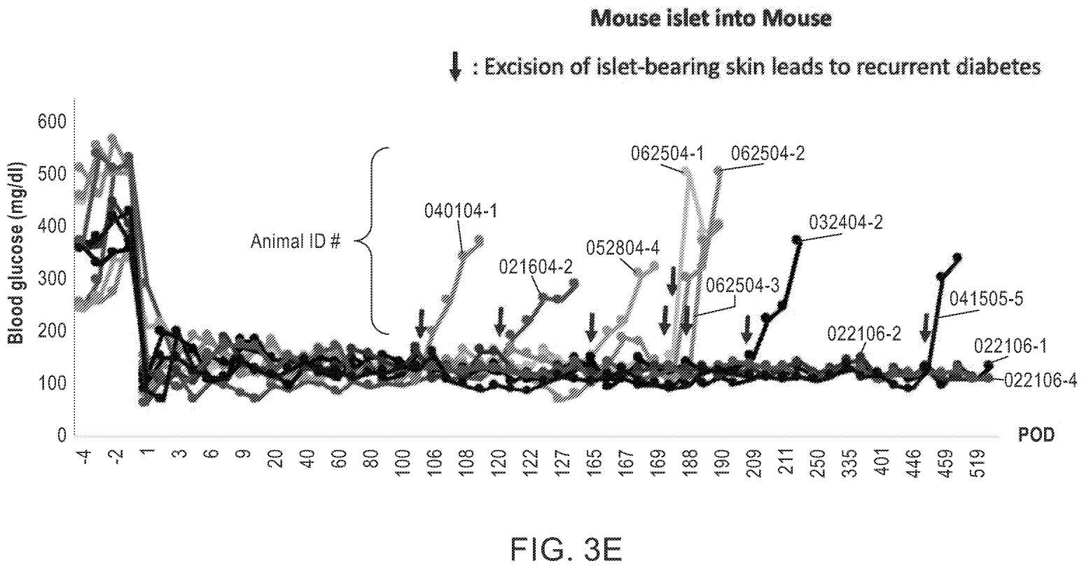

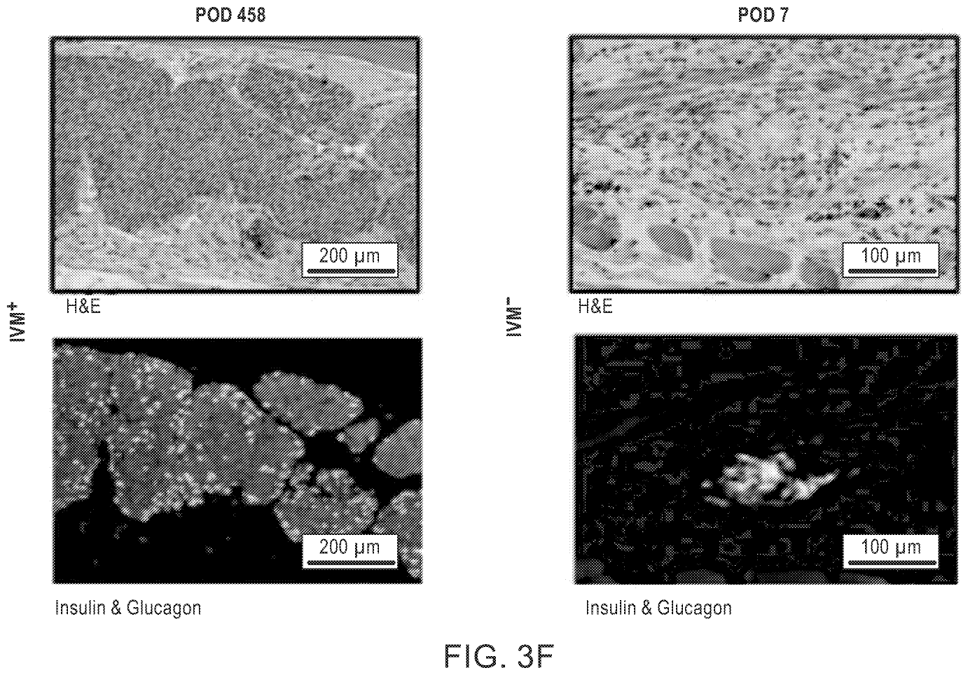

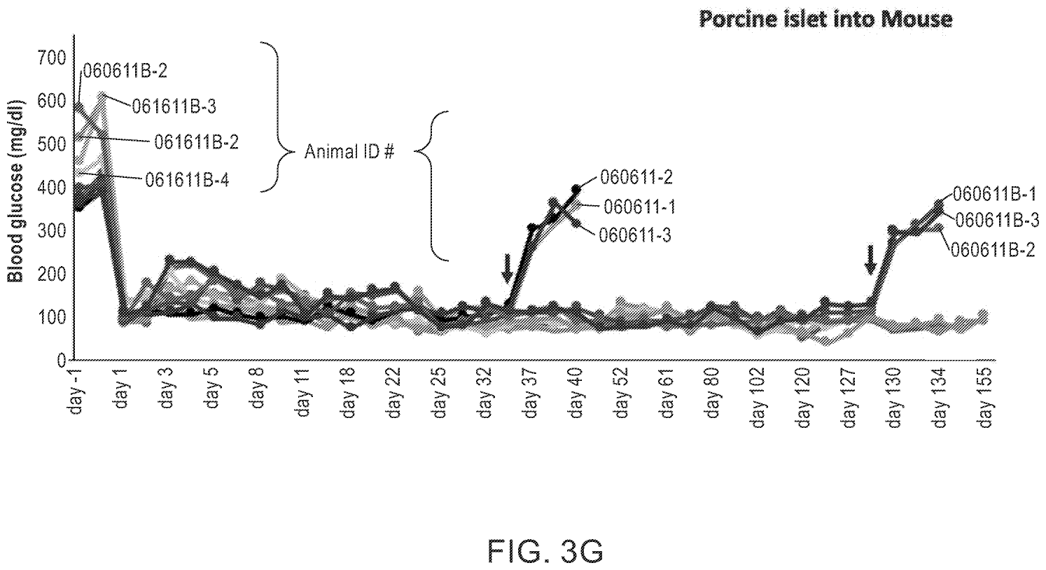

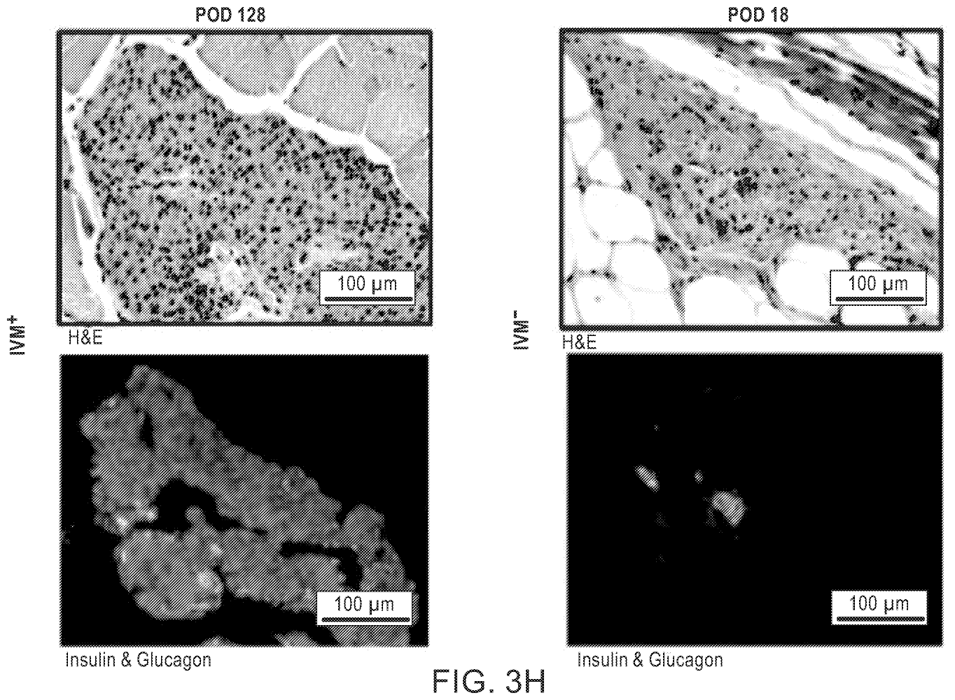

Murine, porcine and human islets admixed with a cell-free IVM (islet viability matrix) and without any device, thrive subcutaneously and maintain robust glycemic control (FIGS. 1A-B). In the murine model, subcutaneous transplantation of 800 syngeneic islets without IVM into diabetic B6 SCID or WT B6 hosts uniformly failed to reverse diabetes, with most experiencing primary non-function (FIG. 1B). In contrast, even a 50% reduction of islet mass bedaubed with IVM rendered the recipients normoglycemic within 24 hours post-transplantation (FIG. 1B). Excision of the islet-bearing skin invariably resulted in recurrent diabetes, and a histologic comparison of the tissue containing IVM-treated (IVM⁺) versus control (IVM⁻) islets demonstrated abundant healthy β-cells in the former case in contrast to loss of islet morphology and viability in the latter cohort (FIG. 3A). These observations were consistent across xenogeneic transplantation of porcine islets (FIG. 1B, FIG. 3C).

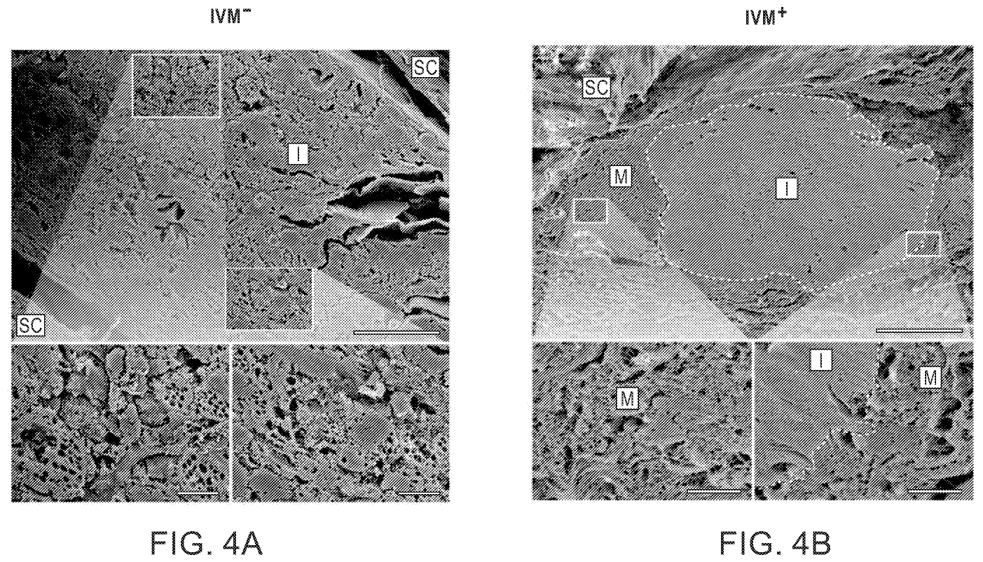

Whether utilizing human islets would afford similar success when engrafted subcutaneously in diabetic immune-incompetent hosts was examined next. In contrast to mice receiving human pancreatic islets transplanted without IVM, those engrafted with IVM uniformly reversed diabetes (FIG. 1C). Excision of the islet-bearing skin in the long-term recipients of human islets led to prompt recurrence of hyperglycemia. The IVM⁺ islets preserved cellular morphology, and interestingly, the collagen matrix in the IVM integrated around the islet cell clusters without being degraded (FIG. 1D). These changes were further substantiated using ultra high-resolution field emission scanning electron microscopy, which unveiled that in the presence of IVM, human islets maintain a healthy morphology, devoid of blebbing or structural degradation (FIG. 4).

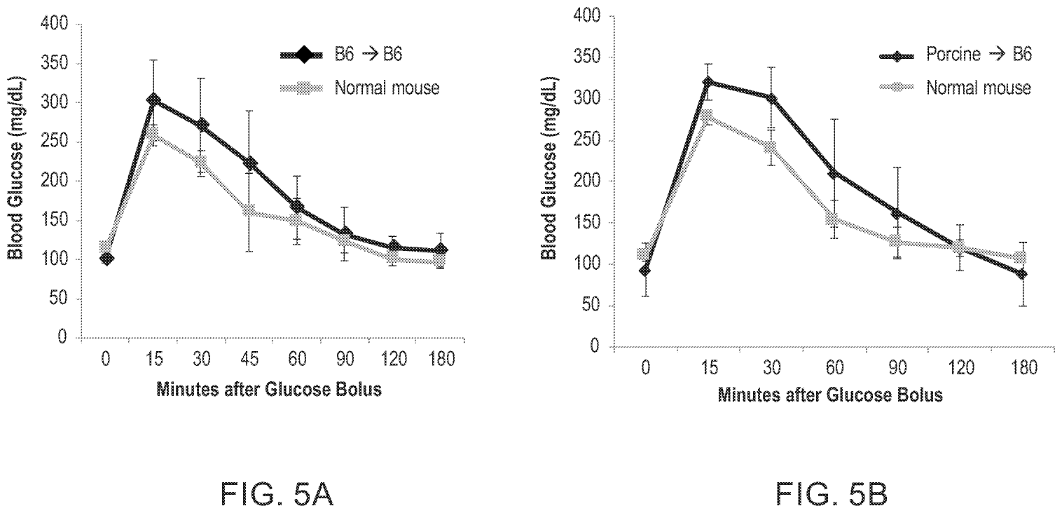

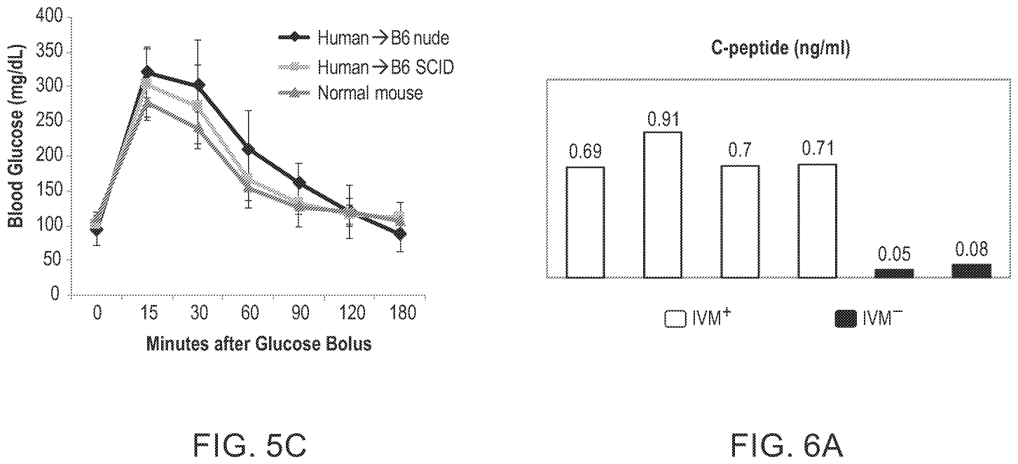

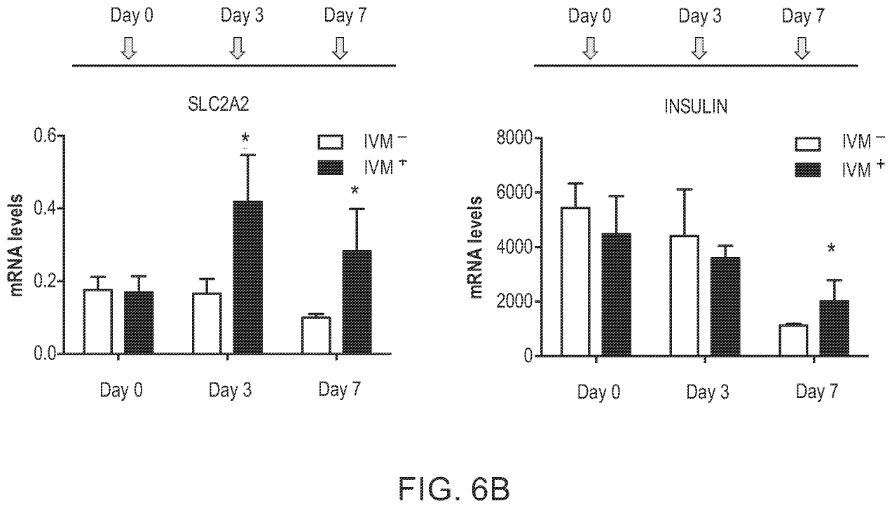

Subsequently, kinetics of glucose disposal was evaluated when the recipients bearing long-term subcutaneous islets underwent glucose tolerance testing. Long-term recipients bearing IVM⁺ islets from mice, swine or humans (n=5 in each group) demonstrated normal glucose disposal, akin to healthy non-diabetic mice (FIGS. 5A-C), further corroborated by human C-peptide level measurements (FIG. 6A). Next, whether IVM induces changes in expression of the primary glucose receptor, Glut2 (SLC2A2), and insulin was examined. To that end, human islets were cultured in the presence or absence of IVM, and a subset of islets was sampled at days 0, 3 and 7. Expression of SLC2A2 and insulin at each time point were quantified and compared between the IVM⁺ and IVM⁻ islets. Although the SLC2A2 and insulin expression levels started out comparably in both IVM⁺ and IVM⁻ islets in culture, at days 3 and 7 the IVM⁺ group demonstrated significantly higher SLC2A2 levels (FIG. 6B; p<0.05). Furthermore, while insulin expression levels fall sharply between days 3 and 7 in the IVM⁻ islets, their levels were persistent in the IVM⁺ group, validating the improved functionality conferred by the IVM.

In anticipation of its translational impact, the above method was corroborated with subcutaneous transplantation of autologous islets in a non-human primate. Grafting macaque islets with IVM underneath the skin rendered the animal normoglycemic (FIG. 1E). Moreover, a biopsy at the islet implantation site on post-operative day (POD) 72 revealed preserved islet morphology, which was sustained even at POD 918 (FIG. 1F). The animal became insulin-dependent on POD 820, likely due to a concordant and progressive increase in body weight, or secondary to reduced islet mass due to a previous excisional skin biopsy.

### Mechanism of Transplant Survival

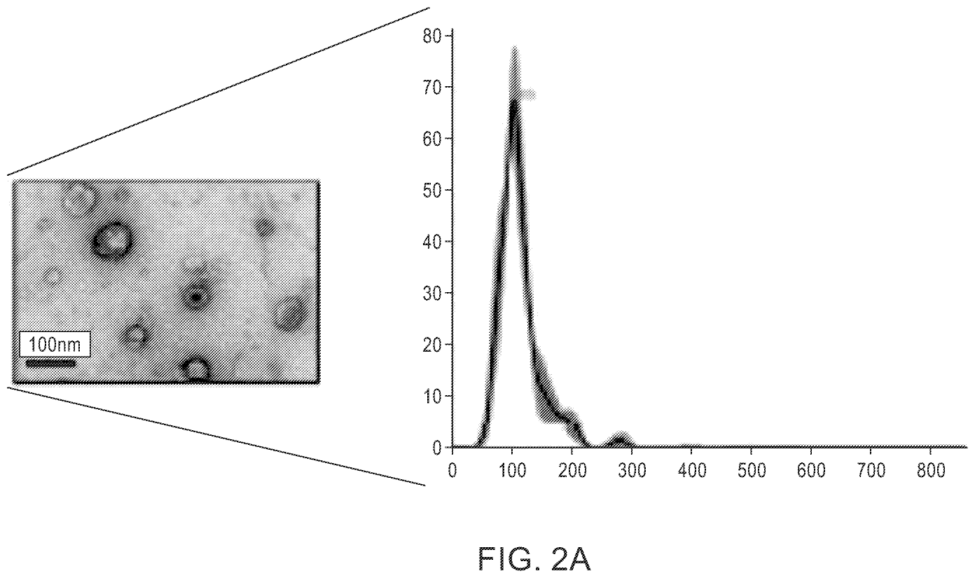

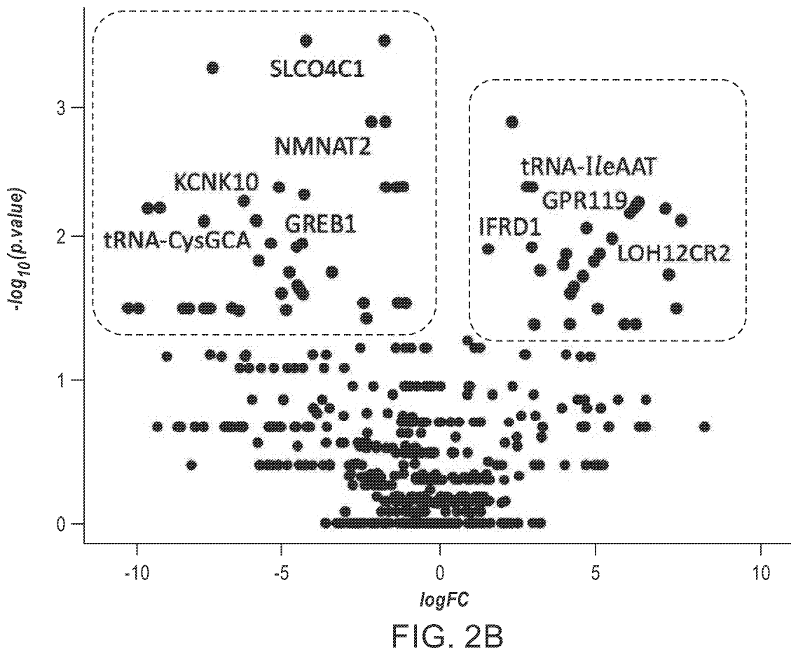

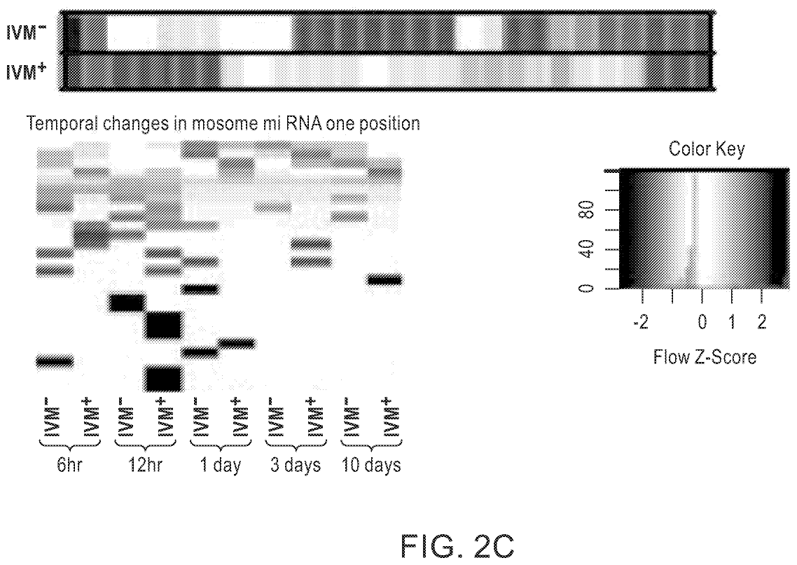

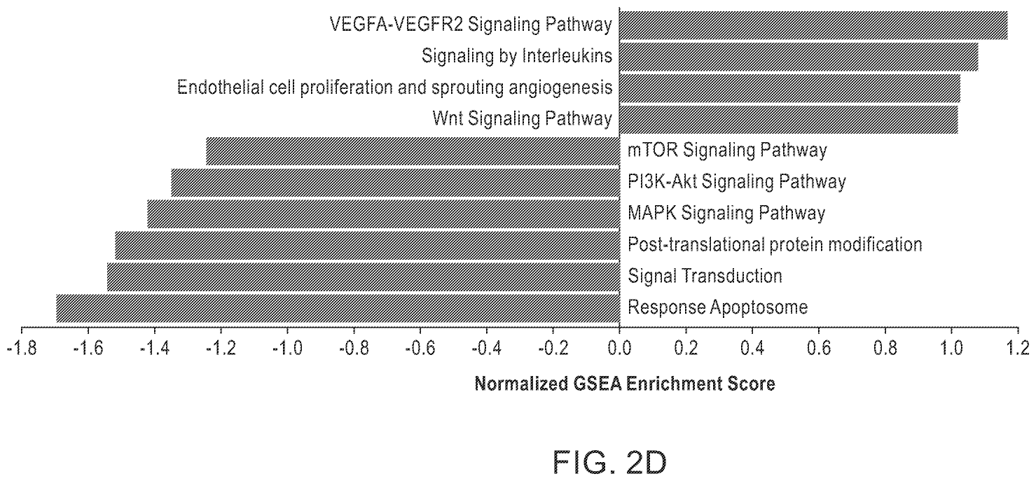

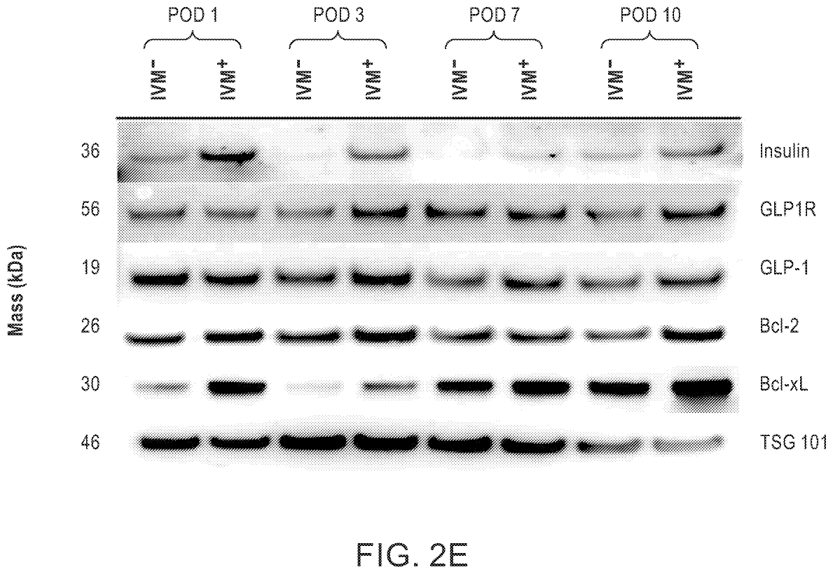

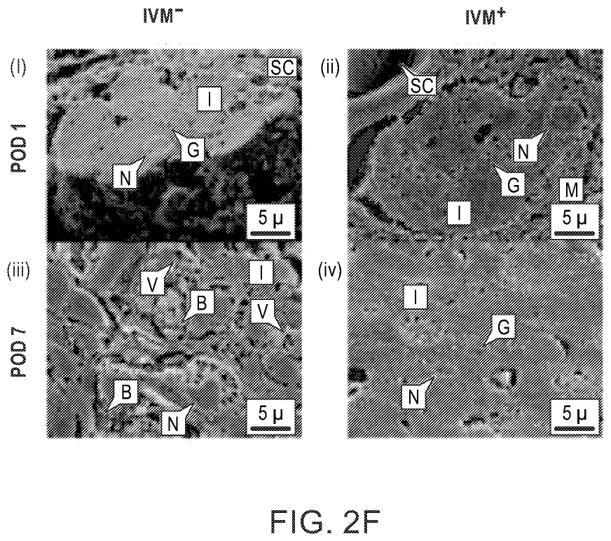

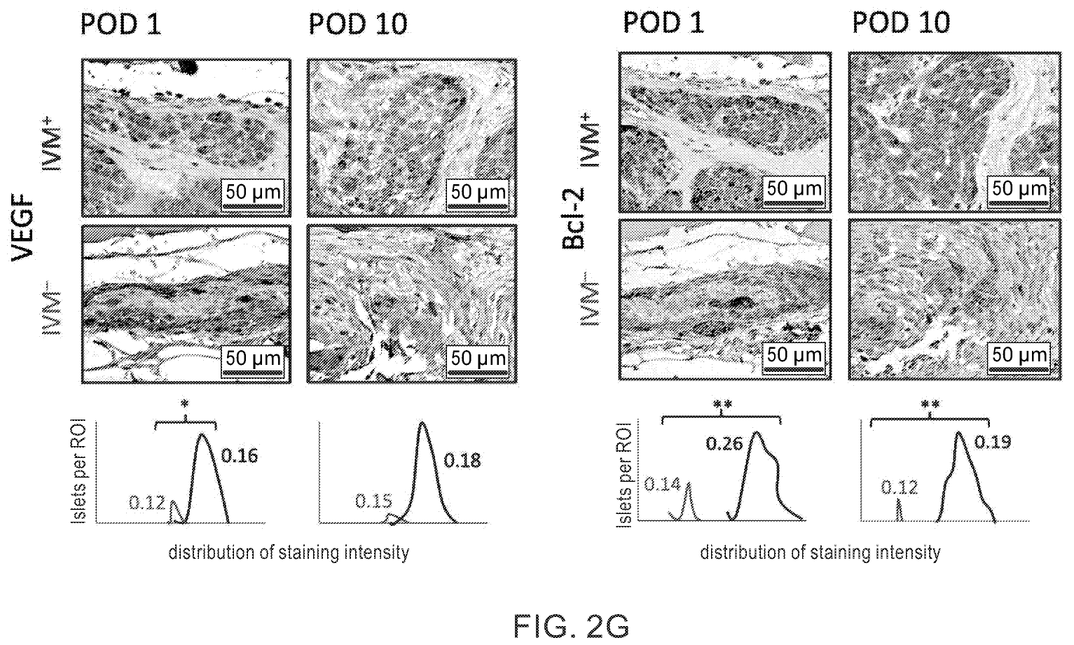

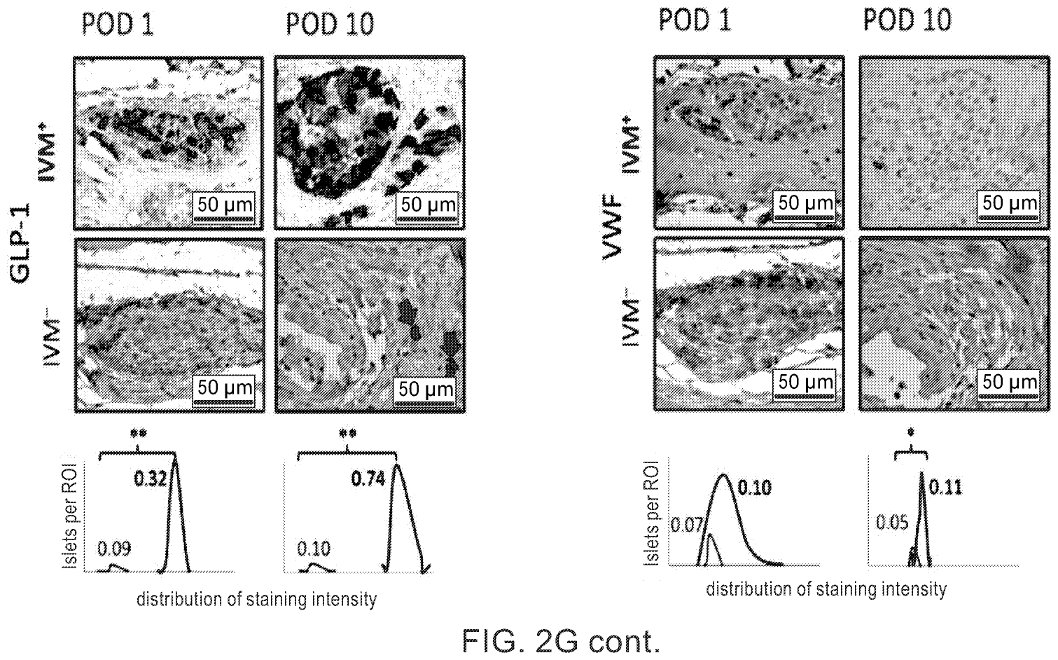

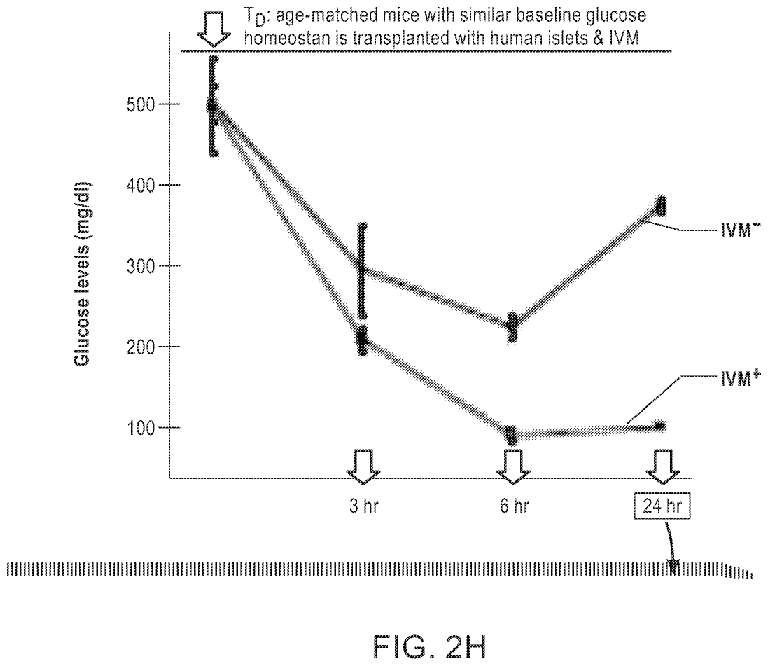

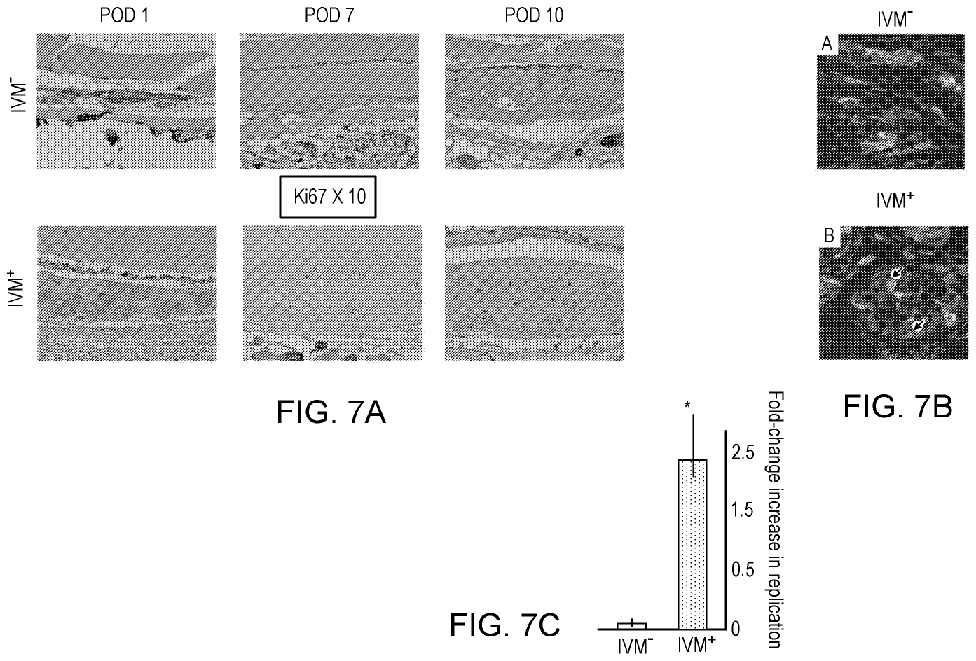

To explore the mechanisms underlying the success of the subcutaneous islet transplant method above, it is hypothesized that the transplant islet-specific exosomes (TISEs) might provide a quantitative window into how IVM fosters cell survival. Thus, the human-into-mouse islet transplant model was utilized and exosomal RNA profiles were compared between the IVM⁺ and IVM⁻ conditions (FIG. 2A) from recipients at different time points during the first 10 days post-engraftment. Comparing the intra-exosomal cargoes between IVM⁺ and IVM⁻ recipients revealed significant upregulation of markers associated with cellular regeneration and insulin regulation (FIG. 2B; p<10⁻³). Additionally, using exosomes as a proxy for the conditional state of the tissue, changes in the grafted human islets with or without IVM were tracked at 6 hr, 12 hr, POD1, POD3 and POD10. It was discovered that several miRNAs such as mir-187, mir-92b and mir-24-2, known to be anti-inflammatory and pro-cell survival, were upregulated in the IVM⁺ group (FIG. 2C). Further, performing an unbiased gene set analysis revealed upregulation of pro-angiogenic and proliferative pathways (FIG. 2D) in the IVM⁺ grafts (IVM⁺ TISEs), whereas apoptosis and mTOR/MAPK/PI3K-Akt pathways were downregulated. These insights, suggesting that the islet-IVM mixture might provoke β-cell replication, were further supported by Ki67 staining and substantiated by increased BrdU incorporation in the presence of IVM (FIG. 7).

Furthermore, western blot analyses showed expression of insulin almost exclusively in the IVM⁺ exosomes, and demonstrated an increased expression of GLP-1, GLPIR, Bcl-2 and Bcl-xL in the IVM⁺ group compared to IVM⁻ (FIG. 2E). The anti-apoptotic Bcl-2 family of proteins play important roles in inhibiting mitochondria-dependent extrinsic and intrinsic cell death pathways, whereas GLP-1 and GLPIR promote pancreatic β-cell function and insulin secretion. Therefore, further evidence was sought for differential apoptosome activity in the grafted islets at an ultrastructural level. Consequently, it was discovered that human islets implanted in subcutaneous tissue without IVM undergo severe apoptotic blebbing and vacuolization post-engraftment. In contrast, islets admixed with IVM retain a healthier phenotype (FIG. 2F). Encouraged by the presence of a pro-survival RNA signature in the IVM⁺ population, these markers were validated at the protein level by immunohistochemistry (IHC).

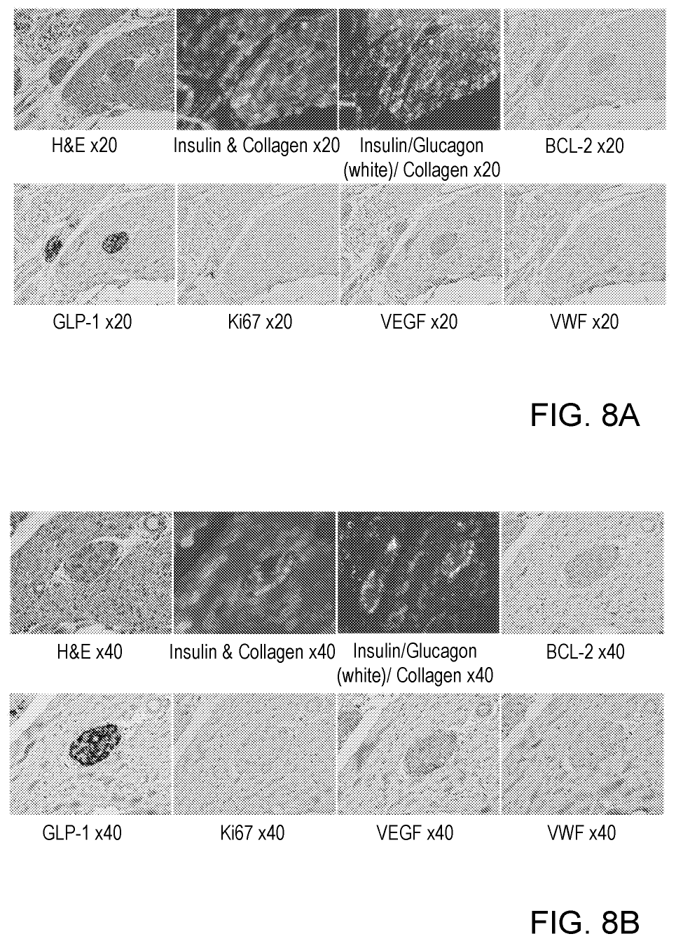

Antibodies targeted at specific epitopes for Bcl-2, Von Willebrand Factor (VWF), Vascular endothelial growth factor (VEGF) and GLP-1 were utilized for staining. The results show that human IVM⁺ islets had a substantially higher proportion and intensity for the stained epitopes compared to IVM⁻ at POD 1 and 10 (FIG. 2G). Additionally, the autologous islet-IVM subcutaneous graft in the nonhuman primate was sampled and it confirmed the presence of these molecules in the macaque islets at POD 918 (FIG. 8). Taken together, these results indicate that enhanced neovascularization, pro-survival signals, and downregulation of the apoptosome might explicate islet survival in the presence of IVM during subcutaneous transplantation.

To better appreciate the temporal dynamics of gene regulation and islet endocrine function, age-matched immunocompetent B6 mice were divided into two groups—in one group, human IVM⁺ islets were subcutaneously engrafted, while the other group received IVM⁻ islets. Glucose measurements at 3, 6 and 24 hr post-transplantation revealed that animals in the IVM⁺ group achieved glucose homeostasis as early as 3 hr post-engraftment (FIG. 2H). Moreover, islet grafts were excised POD 1 from mice in both cohorts to quantify their RNA composition. It is found that SLC2A2 expression was significantly increased (Glut2; p<0.005) in recipients with an IVM⁺ graft (FIG. 2I). In addition to the increased SLC2A2 levels, the IVM⁺ group also demonstrated marked upregulation of insulin, PDX1 and VEGF (FIG. 2I).

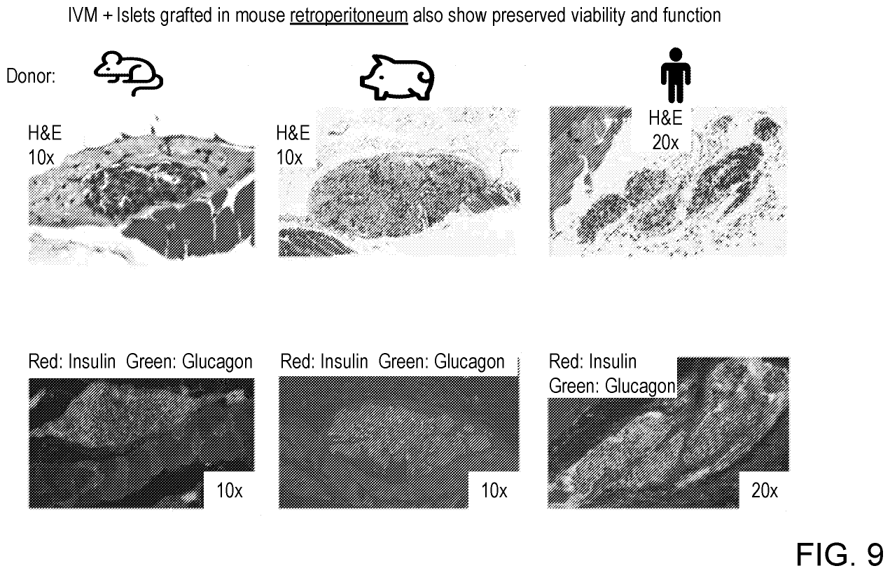

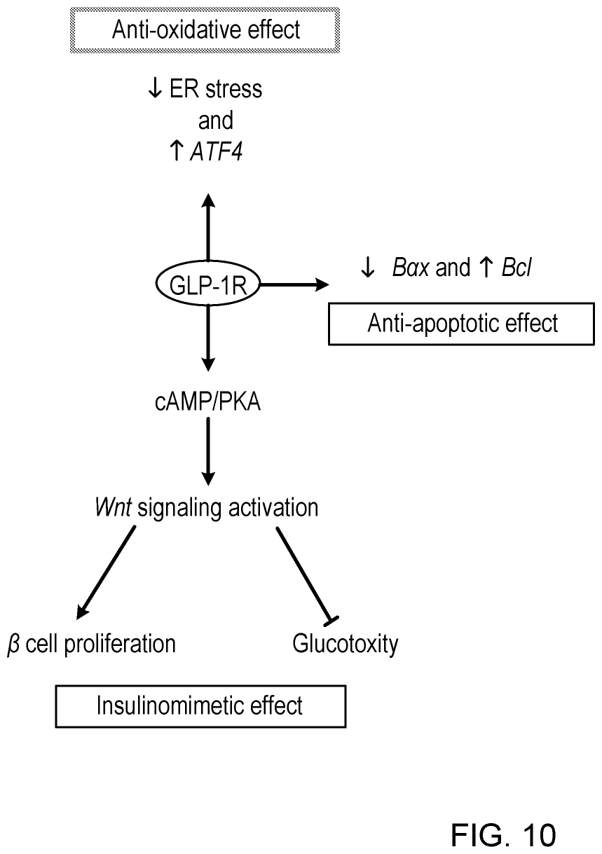

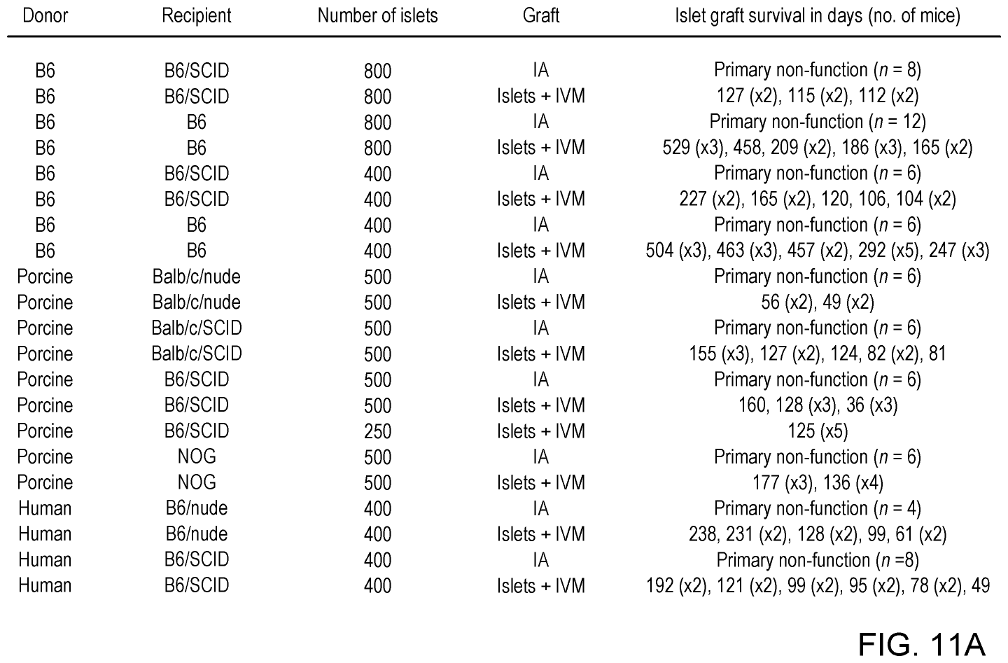

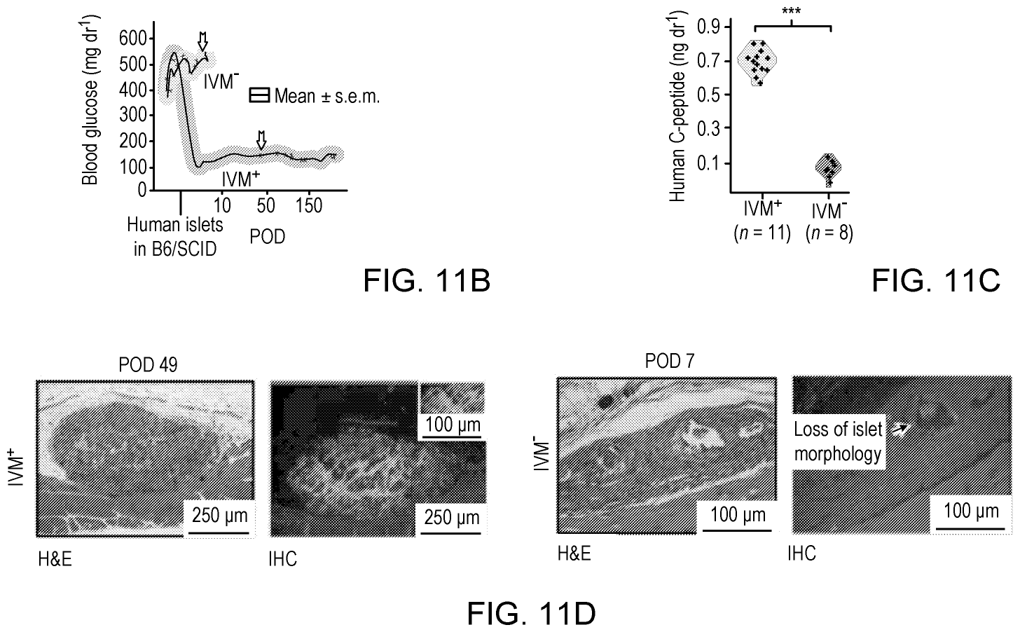

Encouraged by the molecular underpinnings that allow IVM⁺ islets to survive underneath the skin, whether the islet-IVM mixture might enable transplants at other sites, such as the retroperitoneal space, was examined. Indeed, murine, porcine or human islets admixed with IVM and transplanted in the retroperitoneum uniformly restored normoglycemia in diabetic hosts. Moreover, similar to the grafts in the subcutaneous space, excision of the retroperitoneal island containing pancreatic islets led to recurrent diabetes (FIG. 9). These data confirm that IVM enhances islet viability and insulin release in vivo, building on previous work where addition of extracellular matrix components such as collagen could induce islet adhesion and inhibit inflammation. Improved β-cell function in the presence of IVM could also be mediated by the GLPIR signaling pathway. GLP-1 has insulinotropic and insulinomimetic effects on β-cells, mediated in part by enhanced Wnt signaling and anti-oxidative effects. Furthermore, GLP-1R activity has been found to downregulate the pro-apoptotic Bax and upregulate the anti-apoptotic Bcl-2. In line with this notion, upregulation of the Wnt signaling pathway, and a notable paucity of apoptosis, inflammation or fibrosis were found in islets admixed with IVM, thus paving the way for integrative model that expounds the above results (FIG. 10).

### Example 2

### Islet Transplantation in the Subcutaneous Space Achieves Long-term Euglycemia in Preclinical Models of Type 1 Diabetes

This example reports a previously undescribed mixture of human collagen 1, L-glutamine, fetal bovine serum, sodium bicarbonate and medium 199, which when admixed with murine, porcine or human islets, promotes uniform survival of the islets subcutaneously.

### Methods

### Composition and Preparation of the Islet Viability Matrix

10×M199 (Sigma Life Science, Cat #M0650-100ML), L-glutamine (Mediatech Inc., Cat #25-005-CI), FBS (HyClone Laboratories Inc., Cat #SH30071.03) 7.5% sodium bicarbonate, NaHCO₃(Life Technologies Corporation, Cat #25080-094), Type I collagen (Advanced BioMatrix, Cat #5007-20ML)

Table 1 shows the constituents and their respective concentrations needed to create 1.0 mL of Islet Viability Matrix (IVM). Each ingredient and the final product must be kept on ice at all times to prevent solidification at higher temperatures.

TABLE 1
Components of IVM

| | | |
|---|---|---|
| | Concentration in | Approximate |
| Ingredients | 1 ml IVM | Ratio |

| | | | |
|---|---|---|---|
| 10× Medium (M) 199 | 91 | μl | 23 |
| L-glutamine | 8 | μl | 2 |
| Fetal Bovine Serum (FBS) | 100 | μl | 25 |
| NaHCO₃ (7.5%) | 23 | μl | 6 |
| Type 1 Collagen | 778 | μl | 195 |

### Murine Islet Transplantation Models

### Animals

Immune competent (Balb/cByJ—Stock Number: 000651, and C57BL/6J, B6—Stock Number: 000664), and Immune Incompetent (B6.Cg-Prkdc^{scid}/SzJ (B6/scid—Stock Number: 001913), CBySmn.Cg-Prkdc^{scid}/J (Balb/c/scid—Stock Number: 001803), NU/J (Balb/c/nude—Stock No: 002019), B6.Cg-Foxn1^{nu}/J (B6/nude—Stock Number: 000819) and NOD-scid IL2Rgamma^{null} (NOG—Stock Number: 005557)) male mice aged 8-12 weeks, used as islet donors and recipients, were obtained from the Jackson Laboratory, Bar Harbor, ME. Littermate controls were not utilized as these experiments involved transplantation of mouse, porcine and human islets into diabetic recipients.

Animals were housed in conditions to minimize stress, including a 12-hour light/12-hour dark cycle, ˜50% humidity, and a 20-21° ° C. temperature.

### Diabetes Induction

Recipients were rendered diabetic by a single intraperitoneal injection of streptozotocin (SICOR Pharmaceuticals, Inc., Irvine, CA) at a dose of 200 mg/kg. At 5 days after streptozotocin (STZ) administration, animals with three consecutive (daily) non-fasting blood glucose levels>350 mg/dl (Contour Blood Glucose Monitoring System, Bayer HealthCare LLC, Mishawaka, IN, USA) were used as islet recipients. The protocol and all animal studies were approved by the University of Pennsylvania's IACUC (Protocol Numbers: 805662, 800932 and 805005), and in accordance with the NIH's Guide for the Care and Use of Laboratory Animals.

### Islet Isolation

Mouse pancreatic islet isolation was done by collagenase P (Roche Diagnostics, Indianapolis, IN) digestion and density gradient separation. Islets were kept in a suspension of RPMI-1640 medium.

### Islet Transplantation

The recipient mice were anesthetized by inhalation of 2-5% Isoflurane (Isoflurane USP, Clipper Distribution Company LLC, St Joseph, MO, USA).

In the subcutaneous (SC) transplantation model, a small skin incision (0.3-0.5 cm) was established over the lower abdomen to create a right and left lower quadrant SC pocket in which 400 fresh islets (hand-picked) were injected immediately in either a 360 μL suspension of RPMI-1640 (“islets alone”; IA) or admixed in 360 μL IVM into right and left SC pockets separately.

In the retroperitoneal (RP) transplantation model, the mouse right lower abdominal cavity was opened, and 400 manually-picked fresh islets in either a suspension of 180 μL of RPMI-1640 (“islets alone”) or admixed in 180 μL IVM were injected with 25-gauge butterfly needle underneath the peritoneal layer in the right posterior retroperitoneal space.

Of note, regardless of the number of isolated islets (ranging from 200-800), the volume of IVM (360 μL IVM for SC and 180 μL IVM for RP) in which they were suspended remains the same, largely due to anatomical space constraints in the animal.

### Immunosuppressive Regimen Following Allogeneic or Xenogeneic Transplantation in Mice

The immunosuppression regimen was based on targeting both T and B cells to promote the survival of islet allograft tolerance. Briefly, the maintenance immunosuppression consisted of rapamycin (0.5 mg/kg intraperitoneal daily; qd), starting the day of islet transplantation for a duration of seven days. To target the β-cell compartment, we used 10F4, a monoclonal antibody against mouse B lymphocyte stimulator (BLyS), 100 μg intraperitoneally 20 days before transplant in two doses, 24 hours apart. 10F4 was provided by Human Genome Sciences, courtesy of Dr. Michael Cancro (Pathology and Laboratory Medicine, University of Pennsylvania), and eliminates primary B cells. 10F4 was also given again starting day 10 in a tapering dose, from 50 μg/week in week 2, down to 5 μg/week starting week 8. Immunosuppression was discontinued at day 66.

### Porcine Islet Transplantation Models

### Islet Isolation

Porcine islets were obtained from Dr. Bernhard Herring's laboratory at the University of Minnesota. Islets were incubated in CMRL 1066 medium (Mediatech, Inc. Cat #98-304-CV) containing 5.5 mM d-glucose, 0.05% human albumin (Telesis Biotherapeutics, Research Triangle Park, NC), 10 U/mL Heparin (Sagent Pharmaceuticals, Schaumberg, IL), 100 μg/mL penicillin/streptomycin, and 2 mM L-glutamine.

### Islet Transplantation

The recipient mice were anesthetized by inhalation of 2-5% Isoflurane (Isoflurane USP, Clipper Distribution Company LLC. St Joseph, MO). The SC and RP transplantation experiments were carried out as detailed above in the “Murine Islet Transplantation Models” section. The protocol and all animal studies were approved by the University of Pennsylvania's Institutional Animal Care and Use Committee (IACUC) (Protocol Numbers: 805662, 800932 and 805005), and in accordance with the Guide for the Care and Use of Laboratory Animals prepared by the NIH.

### Human Islet Transplantation Models

### Islet Isolation

The comprehensive islet transplantation program at the University of Pennsylvania served as the resource for the human islets used in this study. It is part of the Integrated Islet Distribution Program (IIDP), and along with other centers, formed the Clinical Islet Transplant consortium (CIT) to initiate phase 3 trials of islet transplantation in Type 1 Diabetes. The source (either through IIDP or CIT) of the isolated human islets used in the experiments of this example were all procured per the CIT manufacturing guidelines. which are described at: www.isletstudy.org. Human islets used via both the IIDP program as well as through CIT were procured at University of Pennsylvania's Islet GMP facility. Particularly with respect to the CIT, islets were obtained from pancreata that were originally intended for clinical use as part of the phase 3 clinical islet transplantation study. However, from several donors, the recovered yield was not high enough for clinical use despite high quality of the islets. These islet preps were then used for non-clinical activity such as distribution to research investigators and the experiments described here. The release criteria was followed as mandated by the NIH/FDA, including glucose-stimulated insulin release, restoration of normoglycemia in diabetic NOG mice, and perfusion studies for kinetics of insulin and glucagon secretion.

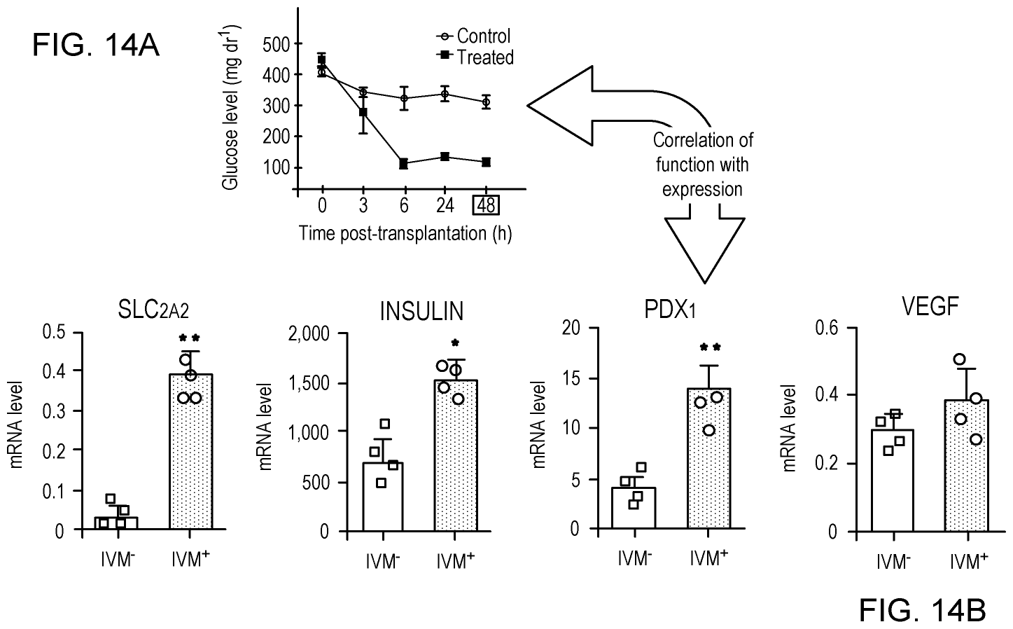

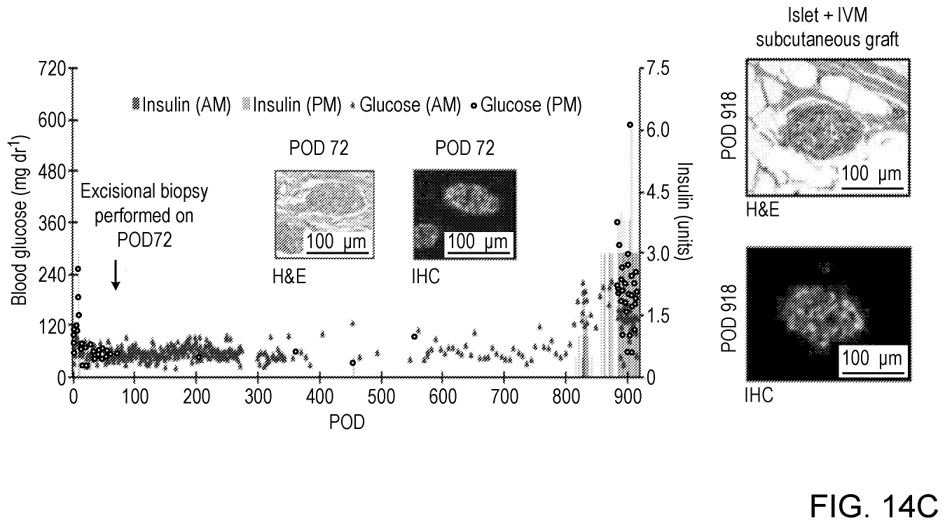

The recovery of pancreata from deceased organ donors was overseen under the auspices of the local/regional organ procurement agency (www.donors1.org; Gift of Life Donor Program, Philadelphia, PA). The staff of the organ procurement agency obtained consent for organ recovery from the next of kin/donor's family members without any intervention from the investigators using the organs for research, in accordance with the U.S. Federal Mandate. A summary of the characteristics of human islet donors used in the experiments is described in FIGS. 1B and 14A.

### Islet Transplantation

The recipient mice were anesthetized by inhalation of 2-5% Isoflurane (Isoflurane USP, Clipper Distribution Company LLC, St Joseph, MO). The SC and RP transplantation experiments were carried out as detailed above in the “Murine Islet Transplantation Models” section.

### Assessment of Islet Graft Function

### Islet Graft Monitoring

Blood Glucose levels were monitored twice daily after transplantation and recipients with non-fasting glucose concentrations<200 mg/dL were considered to have achieved normoglycemia. When two consecutive daily non-fasting glucose levels were >300 mg/dl after a period of one week, islet grafts were considered to have failed and the recipient was considered to have primary non-function of the islet transplant.

### Glucose Tolerance Test

An intraperitoneal glucose tolerance test (IPGTT) was performed on the animals with long-term normoglycemia (>100 days). Blood glucose levels were analyzed using the Contour Blood Glucose Monitoring System (Bayer HealthCare LLC, Mishawaka, IN).

Excision of Islet bearing site

To confirm that the islet grafts were the sole source of maintaining normoglycemia, a cohort of long term normoglycemic islet recipients, underwent excision of the islet bearing sites (skin or retroperitoneum). This uniformly led to prompt recurrence of diabetes within 24 hours. In recipients transplanted without IVM, excision of graft bearing sites occurred on day 7. In recipients transplanted with Islet+IVM, excision of graft bearing sites occurred between 100-458 days.

### Nonhuman Primate Models

### Animals

Two male cynomolgus monkeys (Macaca fascicularis) were obtained from Spring Scientific Perkasie, PA, for autologous islet transplantation. Monkey 1 (ID #212077) had an initial body weight of 3.5 kg and age 4 years, whereas monkey 2 (ID #210069) had an initial body weight of 3.9 kg and age 4 years. Prior to transplantation, serological testing was performed on each animal to ensure that they were not infected with Herpes B. Schmidt-Ruppin strain of the Rous sarcoma virus, Simian T-cell Lymphotropic Virus (STLV), or Simian Immunodeficiency Virus (SIV). All animals had continuous water supply and were fed with regular primate diet supplemented with fresh fruits twice daily. All animal care and handling were done in accordance with the guidelines set forth by the Department of Health and Human Services' guide for care and use of primates, as well as the IACUC guidelines.

### Diabetes Induction

To perform islet auto-transplantation and assess the efficacy of IVM to promote islet engraftment in subcutaneous space in the absence of anti-islet alloreactivity, diabetes in the monkeys was induced by subtotal pancreatectomy (˜85-90%) in 2 cynomolgus monkeys. Monkeys were sedated using ketamine (15 mg/kg) and atropine (0.05 mg/kg), prepared for operation, and intubated. Anesthesia was initiated with midazolam (1 mg/kg) and maintained with isoflurane and oxygen. Haircoat was clipped closely from the nipples to mid-thigh and laterally to the end of the vertebral processes and skin, followed by scrubbing with a preliminary chlorhexidine solution. Animals were placed in a supine position, and the surgical field given a second chlorhexidine scrub. The abdomen was opened using a midline laparotomy. The spleen and greater omentum were reflected to expose the entire pancreas. The right and left pancreatic limbs were mobilized, preserving pancreatic vasculature, and the pancreas then carefully dissected from the duodenal serosa. The common pancreatic duct was identified at the duodenum, ligated, and transversely incised distal to the ligation. Through this incision, pancreatic duct was cannulated with an angio-catheter, and secured with ligature. The pancreas was then excised, placed in cold UW solution and transported to the islet isolation laboratory. Finally, incisions were closed layer by layer. General anesthesia was maintained while the pancreas was processed for islet isolation.

### Islet Isolation

Islets were isolated by collagenase digestion and differential centrifugation.

### Subcutaneous Islet Transplantation

While under general anesthesia, the animals were placed in a supine position, and after the sterile prepping of the abdomen a small incision (3-4 cm) was established in left lower quadrant of the abdomen and the needle connected to a syringe containing the isolated islets plus IVM, was inserted into the subcutaneous space. The total inoculum volume was slowly infused in the subcutaneous compartment and the needle point was then swabbed with an antiseptic. Finally, the animal was recovered in an incubator and extubated thereafter. Per the advice of our IACUC veterinarians, monkeys were not treated with exocrine extracts during the post islet transplantation.

### Islet Graft Function

According to IACUC recommendation, after transplantation blood glucose levels in islet auto-transplanted monkeys were monitored twice daily for the first three months, then daily thereafter. Recipients with non-fasting glucose concentrations<200 mg/dL were considered to have achieved normoglycemia requiring no daily exogenous insulin administration. When two consecutive daily non-fasting glucose levels were >300 mg/dL after a period of primary graft function, islet grafts were considered to have failed mandating treatment with exogenous insulin twice daily. Towards POD 400, as a result of frequent blood sampling from the tip of the tail, the tail developed a wound. The IACUC veterinarian's recommendation was to not sample the animal for a period of several weeks to months, giving it enough time to completely heal. Any gaps in daily blood glucose measurements during the 3-year-long follow up were based on the uniform recommendation of veterinary staff to comply with ethical handling of the animal with proper care.

Biopsies of the islet bearing skin were performed under general anesthesia. Tissue samples (1-2 cm²) were processed for standard H&E and IHC staining.

### Tracking the Clinical Course of the Cynomolgus Monkeys

For cynomolgus monkey #212077 (body weight 3.5 kg), the excised pancreas weighed 6 grams, yielding 84,000 IEQs. The volume of the islet tissue pellet was 0.3 mL which was added to 16.7 mL of IVM yielding an inoculum containing 5,000 IEQ/mL of IVM. The entire inoculum (23,729 IEQ/kg body weight) was infused into the subcutaneous space. It is important to note that the pre-transplant glucose level in this monkey ranged from 30-110 mg/dL. The duration from subtotal pancreatectomy to subcutaneous islet autotransplantation was 5 hours.

For cynomolgus monkey #210069, in addition to the protocol detailed above, this animal received STZ (50 mg/kg) after subtotal pancreatectomy to eliminate residual native islets in the pancreatic head remnant. The excised pancreas weighed 5 gm, yielding 46,125 IEQ's that were cultured in vitro overnight. During this period, the monkey had free access to water and food and several blood glucose determinations revealed the level 200 mg/del requiring exogenous insulin treatment. The islets were then removed from the culture. The islet tissue pellet volume was 0.2 mL which was added to 9.8 mL of IVM, yielding an inoculum containing about 5,000 IEQ/mL of IVM. The entire inoculum (11,827 IEQ/kg body weight) was transplanted subcutaneously into the LLQ of the abdomen. Of note, the duration from subtotal pancreatectomy to subcutaneous islet autotransplantation was 20 hours. In our attempts to eliminate the residual islets in the remnant of the pancreatic head, persistent STZ in the animal may have led to the loss of islet cells transplanted under the skin 20 hours after STZ administration.

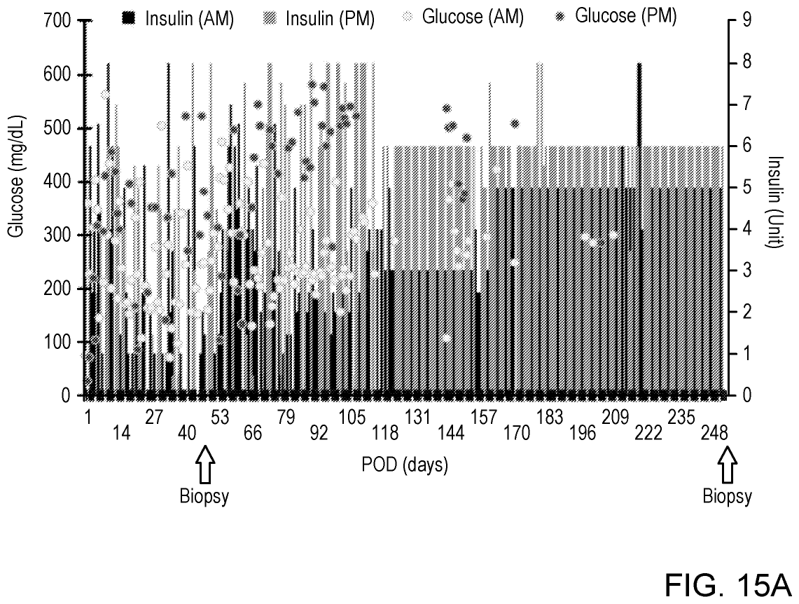

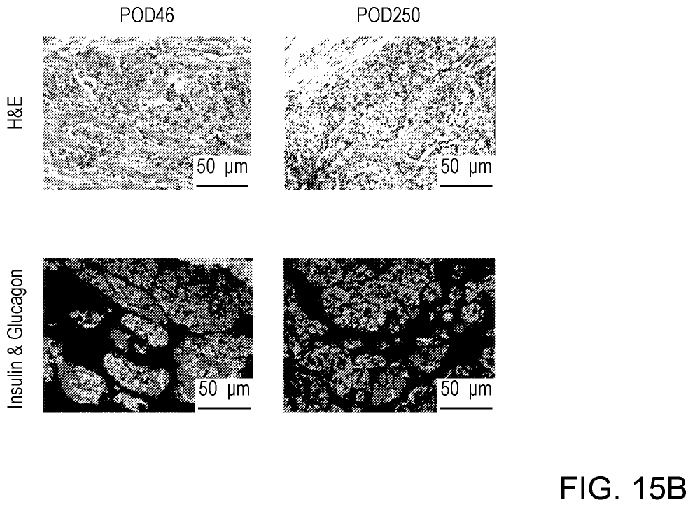

During a year-long follow up, the animal remained diabetic, requiring daily insulin (FIG. 15A). Biopsy of islet-bearing skin at POD 46 and 250 demonstrated healthy, insulin- and glucagon-positive islets at the transplant site, without peri-islet fibrosis or any mononuclear infiltration (FIG. 15B). Despite the maintenance of islet morphology, this animal was not rendered euglycemic likely due to three factors. First, islet yield from this monkey was significantly lower. Second, there was inevitable attrition of islets in culture. Third, streptozotocin may have led to β-cell glucotoxicity and subsequent loss of the transplanted islet cells in vivo, although streptozotocin kinetics have been previously studied in rodent models. Nonetheless, these data collectively support the notion that IVM allows long-term persistence of islet viability subcutaneously, and that this protective effect appears to be mediated, at least in part, by upregulation of anti-apoptotic signaling.

### BrdU Incorporation Assay

In vivo bromodeoxyuridine (BrdU) labeling was achieved by diluting drinking water with (1 mg/mL) BrdU (Product #B9285, Sigma-Aldrich Co., LLC. St. Louis, MO) for 3 days, as previously described. Briefly, islet bearing skin sections containing human islets were harvested, fixed overnight with 4% paraformaldehyde, and processed for paraffin sectioning. Histological analysis of slides was performed using BrdU, Ki67, insulin, and glucagon. Slides were processed for immunostaining as follows: sections were incubated in blocking reagent (1% BSA in PBS) for 30 min, followed by incubation with the appropriate primary antibodies in the blocking reagent overnight at 4° C. Slides were washed thrice in PBS for 3-5 min each, followed by incubation for 45 min at room temperature in the appropriate secondary antibodies in blocking reagent. Slides were washed again in PBS (thrice for 5 min each) and mounted. Images were captured on the Keyence All-in-One Fluorescence Microscope using the 0.95 NA 40× objective (Nikon). BrdU-positive nuclei were counted, blinded from at least 20 islet β-cells per mouse from four mice per group. The protocol was approved by the IACUC of the University of Pennsylvania.

### RNA Isolation for Real-Time (RT) PCR Analysis

Islets from overnight fasted 18-wk-old mice were isolated using the standard collagenase procedure. Islet purity was assessed by Dithizone staining and shown to be >90% endocrine tissue. Total RNA from islets was isolated in Trizol (Invitrogen) followed by RNeasy mini kit or with RNeasy FFPE kit according to the manufacturer's instructions. Islet RNA was reverse transcribed using either 1 μg of Oligo(dT) primer or random hexamer, SuperScript II Reverse Transcriptase, and accompanying reagents (Invitrogen). PCR reactions were assembled using the Brilliant SYBR Green QPCR Master Mix and done with the SYBR Green (with Dissociation Curve) program on the Mx3005P qPCR System (Stratagene). All reactions were done in triplicate with reference dye normalization, and median CT values were used for analysis. The following primers were used:

hSLC2A2 F:
(SEQ ID NO: 13)
ATCCAAACTGGAAGGAACCC
hSLC2A2 R:
(SEQ ID NO: 14)
CATGTGCCACACTCACACAA
hINSULIN F:
(SEQ ID NO: 15)
AGGCCATCAAGCAGATCACT
hINSULIN R:
(SEQ ID NO: 16)
GCACAGGTGTTGGTTCACAA
hPDX1 F:
(SEQ ID NO: 17)
CCTTGTGCTCGGGTTATGTT
hPDX1 R:
(SEQ ID NO: 18)
ATCATCCCACTGCCAGAAAG
hVEGF F:
(SEQ ID NO: 19)
CTACCTCCACCATGCCAAGT
hVEGF R:
(SEQ ID NO: 20)
GCAGTAGCTGCGCTGATAGA

### Immunohistochemistry Analyses

Islet-bearing skin or native pancreatic tissue biopsy samples were fixed in Bouin's or formalin solution. The tissues were processed for routine histology and stained with hematoxylin and eosin (H&E). For immunohistochemical analysis, serial paraffin sections were prepared and stained using anti insulin and glucagon (DAKO Cytomation, Carpinteria, CA), anti-Bovine and human collagen I, anti-human Bcl-2, anti-human VWF, anti-human VEGF, anti-human GLP-1 (Abcam, Cambridge, MA) and anti-human Ki67 antibodies (ThermoScientific, Grand Island, NY). The anti-collagen antibodies were species specific with minimal to no cross-reactivity with mouse collagen. The following products were used: anti-human collagen antibody (Product #C-2456, Sigma, Saint Louis) and anti-bovine collagen antibody (NB100-64523, Novus Biologicals).

Immunofluorescence Antibodies conjugated with Cy2 or Cy3 IgG (Jackson IRL, West Grove, PA) were used as the second step reagents. Immunohistochemistry for Bcl-2, VWF, VEGF, GLP-1 staining was carried out using the Dako Envision+system, peroxidase diaminobenzidine method (Dakocytomation, Carpinteria, CA). After de-paraffinization, antigen retrieval was carried out by boiling the slides in 10 mM citrate buffer, pH 6 or tris-based, pH 9 (Vector, Burlingame, CA) for 20 min. All antibodies at optimal dilution were incubated overnight at 4° C. Slides were then incubated with anti-rabbit or mouse HRP polymer for 30 min at room temperature followed by DAB+substrate-chromagen solution for 5 min at room temperature. Slides were counterstained with hematoxylin and mounted. The details of all the antibodies used for immunohistochemistry are described in Table 2.

TABLE 2
Details of antibodies used for immunohistochemical
analyses of the subcutaneously transplanted islets

| | | Catalog Number (Clone, | | |
|---|---|---|---|---|
| Antibody | Vendor | where applicable) | Lot Number | Dilution* |
| Polyclonal guinea pig | Dako | A0564 | 10095957 | 1:250 |
| anti-Insulin | | | | |
| Polyclonal rabbit anti- | Proteintech Group, Inc | 15954-1-AP | 00024282 | 1:500 |
| Glucagon | | | | |
| Monoclonal mouse anti- | Sigma | C2456 (COL-1) | 088M4784V | 1:75 |
| Collagen I | | | | |
| Monoclonal rabbit anti- | Abcam | ab32124 (E17) | GR3232704-3 | 1:100 |
| Bcl-2 | | | | |
| Monoclonal mouse anti- | Abcam | ab201336 | GR3195786-9 | 1:100 |
| VWF | | (3E2D10 + VWF635) | | |
| Monoclonal rabbit anti- | Abcam | ab27620 (SP28) | GR3214982-1 | RTU |
| VEGF | | | | |
| Monoclonal rabbit anti- | Abcam | ab108443 | GR215906-3 | 1:100 |
| GLP-1 | | (EPR4042-1) | | |
| Monoclonal rabbit anti- | Thermo Fisher | RM-9106-R4 (SP1) | 9106R802C | RTU |
| Ki67 | Scientific | | | |
| Cy ™2 AffiniPure | Jackson | 711-225-152 | 136061 | 1:400 |
| Donkey Anti-Rabbit IgG | ImmunoResearch | | | |
| (H + L) | Laboratories, Inc | | | |
| Cy ™2 AffiniPure | Jackson | 715-225-151 | 137363 | 1:400 |
| Donkey Anti-Mouse IgG | ImmunoResearch | | | |
| (H + L) | Laboratories, Inc | | | |
| Cy ™3 AffiniPure | Jackson | 706-165-148 | 119998 | 1:400 |
| Donkey Anti-Guinea Pig | ImmunoResearch | | | |
| IgG (H + L) | Laboratories, Inc | | | |
| Cy ™5 AffiniPure | Jackson | 711-175-152 | 139802 | 1:200 |
| Donkey Anti-Rabbit | ImmunoResearch | | | |
| IgG | Laboratories, Inc | | | |
| Coat anti rabbit HRP | Vector lab | MP-7451 | ZE1015 | RTU |
| conjugated antibody | | | | |
| Coat anti mouse HRP | Vector lab | MP-7452 | ZE0620 | RTU |
| conjugated antibody | | | | |

*RTU indicates ready-to-use; no dilution needed.

The proportion and intensity of the stained epitopes were evaluated using QuPath v0.1.2. Before running the image analysis algorithm on a particular slide, automated image masking was done per the developer's recommended steps (github.com/qupath/qupath/wiki). The accuracy of this automated ROI annotation was manually checked by an independent pathologist. Subsequently, QuPath's algorithm analyzed all ROIs in the inclusion annotation, excluding any tissue artifacts. Each section image had ≥5 ROIs of sufficiently high quality, and using each ROI as the unit of analysis, a Kruskal-Wallis H Test was performed to compare the output distributions for the proportion and intensity of staining between the IVM⁺ and IVM⁻ groups.

### Biochemical Analysis of Transplanted Islet-Specific Exosome Contents

### Affinity Antibody-Coupled Bead Purification of Tissue Specific EVs

MHC specific antibody was covalently conjugated to N-hydroxysuccinamide magnetic beads (Pierce) per manufacturer's protocol. 50 to 100 μg protein equivalent of EVs were incubated with antibody beads overnight at 4° C. The bead bound EV fractions were separated per manufacturer's protocol. EVs bound to beads were eluted using tris glycine and utilized for downstream analysis. Unconjugated HLA allele-specific anti-HLA A2 monoclonal IgG antibody (Catalogue #0791HA) was purchased from One Lambda (West Hills, CA), for donor HLA class I specific exosome isolation from recipient mouse plasma total pool of exosomes. Antibodies to insulin (15848-1-AP; used at a dilution of 1:200), TSG 101(28283-1-AP; used at a dilution of 1:500) were purchased from Proteintech Lab; antibodies to GLPIR (sc-390774; used at a dilution of 1:200), GLP-1 (sc-57166; used at a dilution of 1:200), Bcl-2 (sc-7382; used at a dilution of 1:200), and Bcl-XL (sc-56021; used at a dilution of 1:200) were purchased from Santa Cruz Biotechnologies. Secondary antibodies conjugated to HRP (ready-to-use anti-rabbit, anti-mouse were purchased from Vector Lab: MP7451 and MP7452, respectively).

### Extracellular Vesicle (EV) Isolation

Exosomes were isolated from mouse plasma by using Sepharose 2B bead based size exclusion chromatography followed by ultracentrifugation. Briefly, 250 μL to 500 μL of plasma was passed through a Sepharose 2B column. Eluent was collected in fractions and pooled after monitoring absorbance at 280 nm. The pooled fraction was ultracentrifuged at 110,000 g for 2 hours at 4° C., pellet was resuspended in 1×PBS for downstream analysis. Purified nanoparticles were analyzed on the NanoSight NS300 at light scatter mode for exosomes quantity and size distribution according to manufacturer's protocols (Malvern instruments Inc., MA).

RNA Isolation from Exosomes.

Total RNA (including microRNAs and mRNA), was extracted from NHS-HLA-Class I bead bound exosomes using Trizol, followed by RNeasy mini kit, according to manufacturer's protocol (Qiagen, Germany).

### Western Blot Analysis.

Donor HLA-class I specific exosomes bound to NHS-beads were lysed and separated on polyacrylamide gels, and transferred onto Nitrocellulose membrane (Life Technologies, NY). The blot was blocked, incubated with desired primary antibody, HRP coupled secondary antibody (Santa Cruz Biotechnologies) per manufacturer's protocol and detected through Chemiluminescence using Phospho-Imager (Amersham Imager 680, GE Health).

### Library Preparation and Sequencing

Exosomal RNA samples were assayed for quantity and quality with an Agilent 2100 Bioanalyzer instrument using the Agilent RNA 6000 Pico Kit (Agilent Technologies, Part number 5067-1513). Libraries were prepared using QIAseq miRNA Library Kit (QIAGEN, cat #331502) as per standard protocol in the kit's sample prep guide. Libraries were assayed for overall quality and quantified using High Sensitivity DNA Kit of Agilent 2100 Bioanalyzer (Agilent Technologies, Part number 5067-4626). Samples were multiplexed for sequencing. 100 bp single-read sequencing of multiplexed pool of samples was carried out on an Illumina HiSeq 4000 sequencer. Illumina's bcl2fastq version v2.20.0.422 software was used to convert bcl to demultiplexed fastq files.

### Trimming Adapter Sequence and UMI Extraction

The library prep kit when sequenced to 100 bp produces reads a the read, a UMI, as well as fixed or nearly fixed sequences. The program cutadapt was used to remove the trailing adapter “AGATCGGAAGAGCACACGTCT” with settings -m 36 -max-n 1. Then the UMI was extracted and the putative smRNA sequence using a custom R script. Briefly, trimmed reads that had more than 3 Ns, or were less than 55 bases long, or did not contain an exact match to the sequence “AACTGTAGGCACCATCAAT” were dropped. The last 12 bases of reads were trimmed and recorded as the UMI. The 19 bases matching the inner adapter sequence were trimmed and the leading sequence was retained as the smRNA sequence. The UMI was appended to the def-line, and the trimmed read and base qualities were saved in FASTQ format.

### Building Chimeric Bowtie Libraries and Transcript Quantification

Three bowtie libraries (bowtie v1.2.3) were built that consisted of mouse and human (1) miRNA hairpins, (2) tRNAs, and (3) RefSeq sequences. The smRNA reads from above were aligned to each separately using the command ‘bowtie -q -k 4 --best --sam --norc’. Expression of miRNA, tRNA, and RefSeq were quantified using the bowtie output files using a custom R script that used libraries ‘Rsamtools’, and ‘GenomicAlignments’ to process the bowtie BAM files. Simple species filtering and UMI reduction was performed as follows. Alignments were filtered to retain only those with the best bowtie stratum (XA BAM tag). Then any reads which had a top-stratum match to both human and mouse were discarded. UMI reduction was performed by binning unique combinations of reference sequence name (i.e., miRNA, tRNA, or RefSeq id), start position, and UMI. Overall raw expression for a reference sequence was calculated as the number of unique reads aligning to each sequence. Raw read counts were also converted to CPM by adding a pseudocount of 1 to all detected transcripts, normalizing to total counts, then taking log 2.

### Gene Expression in Immunodeficient Mice

To further examine the kinetics of gene expression with islet endocrine function in vivo, further to the study in immunocompetent B6 mice in Example 1, age-matched streptozotocin-treated NSG mice were divided into two groups—in one group, human IVM⁺ islets were subcutaneously engrafted, while the other group received IVM⁻ islets. Glucose measurements at 3, 6, 24 and 48 hr post-transplantation revealed that animals in the IVM⁺ group achieve glucose homeostasis as early as 6 hr post-engraftment (FIG. 14A). RNA analysis from human islet grafts excised at POD 2 from mice in both cohorts substantiated a significant increase in SLC2A2 expression (p=0.002) in recipients with an IVM⁺ graft, and there was marked upregulation of SLC2A2, INSULIN and PDX1 (FIG. 14B). These insights, suggesting enhanced islet cell survival and replication in the presence of the islet-IVM mixture, were further supported by increased BrdU incorporation in the presence of IVM (FIG. 7B-7C).

TABLE 3
Results of human IVM⁺ islet or IVM⁻ islet transplantation in
NSG immunodeficient mice and immunocompromised B6/Nude mice.

| | N/sample | Number | Transplant | | | | |
|---|---|---|---|---|---|---|---|
| Recipient | size | of Islets | Site* | Graft^{γ} | NG₂₄ | NG₄₈ | NG₁₆₈ |
| NSG | 8 | 200 | KC | lA | 0 | 0 | 25 |
| NSG | 6 | 200 | PV | lA | 0 | 0 | 17 |
| NSG | 6 | 200 | SC | lVM | 17 | 33 | 67 |
| NSG | 10 | 400 | KC | lA | 60 | 60 | 80 |
| NSG | 6 | 400 | PV | lA | 33 | 50 | 67 |
| NSG | 8 | 400 | SC | lVM | 88 | 100 | 100 |
| B6/Nude | 10 | 500 | KC | lA | 60 | 70 | 100 |
| B6/Nude | 5 | 500 | PV | lA | 60 | 60 | 80 |
| B6/Nude | 10 | 500 | SC | lVM | 80 | 80 | 100 |

*KC = Kidney Capsule; PV = Portal Vein; SC = Subcutaneous Space.
^{γ}lA = islets alone; lVM = islets implanted subcutaneously with lVM.
NGₓ = % of animals normoglycemic within x hours
NSG = non-obese diabetic SCID gamma mouse

### Statistics and Reproducibility

All statistical analyses were performed using R v3.5.3 (www.r-project.org). Data are expressed as mean±standard deviation unless indicated otherwise. The significance of differences between two independent groups was calculated by the Wilcoxon Rank Sum/Mann-Whitney U Test, or the Student's two-sample t-test, as indicated. For representative IHC images, experiments were performed five independent times using distinct biological isolates. The IHC distributions obtained from QuPath were compared between the IVM⁻ and IVM⁺ groups using the Kruskal-Wallis H Test. Cell culture experiments were repeated in independent biological triplicates to ensure reproducibility of the observations. Differences were considered significant at P<0.05 after Benjamini-Hochberg correction for multiple hypothesis testing. The initial pathway analyses were carried out using multicross (cran.r-project.org/web/packages/multicross/index.html), and subsequent gene-set enrichment analysis performed using the WEB-based Gene SeT AnaLysis Toolkit (WebGestalt; www.webgestalt.org). A filter of False Discovery Rate (FDR)<0.05 was used to obtain selected pathways with the highest enrichment.

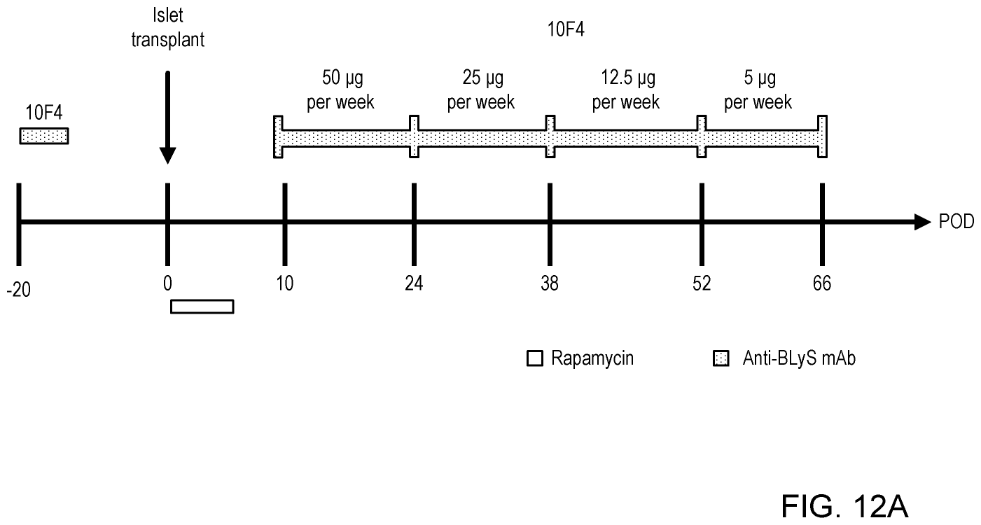

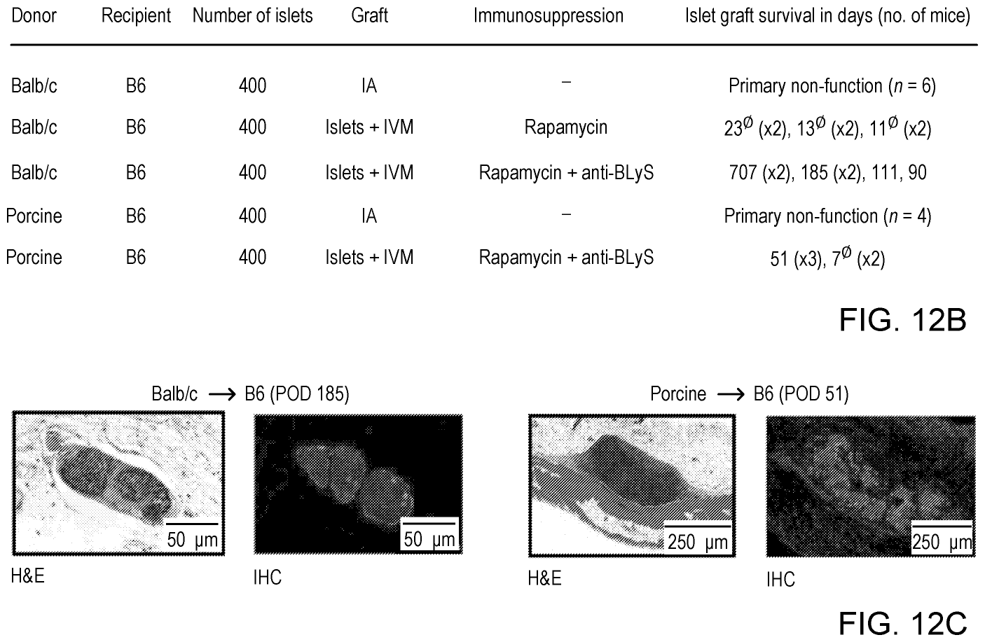

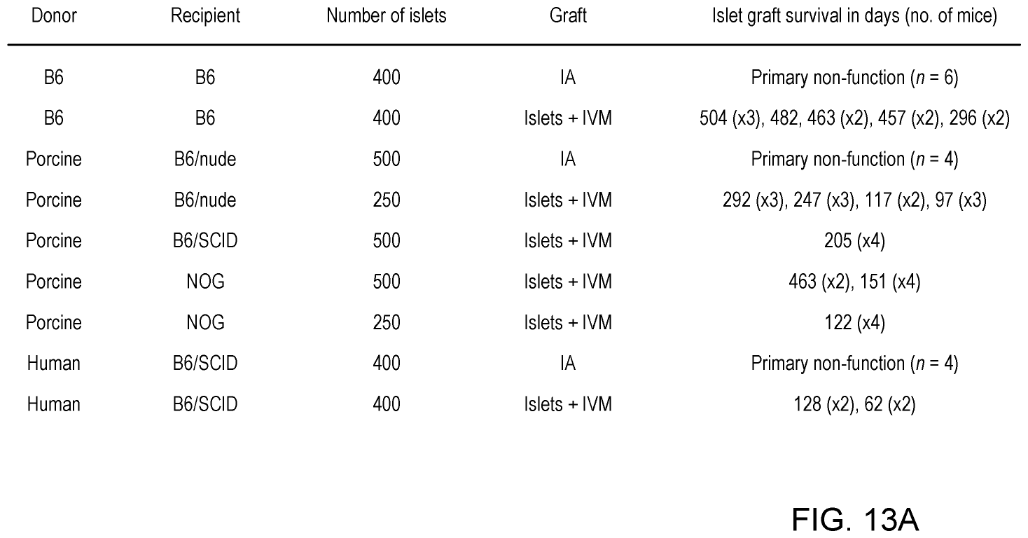

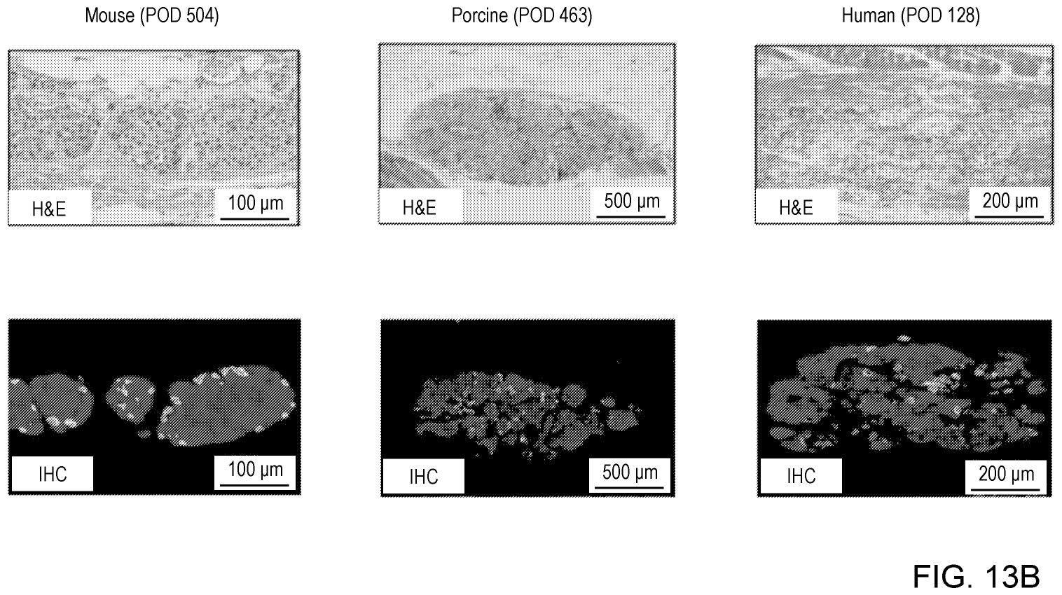

Unless stated otherwise, each experiment was repeated a minimum of five times per sample. Representative histology images shown in FIGS. 1D, 1F, 12C, 13B and 14C, as well as in FIGS. 2G, 3F, 3H, 8A-8B and 15B. Similar results were obtained each of the five times the histologic characterization of the grafts was done for each sample.

### Data Availability Statement

Raw and processed exosome sequencing data have been submitted to GEO (Accession number GSE145593).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition to facilitate the survival and engraftment of cells for transplantation, comprising: collagen I, albumin, L-glutamine and NaHCO₃.

2. The composition of claim 1, wherein the cells are human cells or non-human animal cells.

3. The composition of claim 2, wherein the cells are β-cells of pancreatic islets.

4. The composition of claim 2, wherein the cells are endocrine and/or secretory cells selected from the group consisting of hepatocytes, parathyroid cells and adrenal gland cells.

5. (canceled)

6. The composition of claim 3, wherein the β-cells are human β-cells, murine β-cells, or porcine β-cells or are stem cell-derived β-cells.

7. The composition of claim 4, wherein the endocrine cells are alpha cells or stem cell-derived endocrine cells.

8.-10. (canceled)

11. The composition of claim 1, wherein the composition comprises about 60% to 94% of human collagen I, about 4% to 15% of human albumin, about 0.4% to 4% of L-glutamine, and about 1% to 5% of NaHCO₃.

12.-14. (canceled)

15. A method of transplanting β-cells of pancreatic islets into a host site, comprising:
(a) mixing a population of the β-cells with the composition of claim 1; and
(b) transplanting the cells from (a) into the host site.

16. The method of claim 15, wherein the host is a human or a non-human animal.

17. The method of claim 15, wherein the β-cells are human β-cells, murine β-cells, or porcine β-cells or are stem cell-derived β-cells.

18. (canceled)

19. The method of claim 15, wherein the β-cells are autologous.

20. The method of claim 15, wherein the host is a human and the β-cells are non-human β-cells, wherein the non-human β-cells are murine β-cells or porcine β-cells.

21. (canceled)

22. The method of claim 15, wherein the site is a subcutaneous space, a retroperitoneal space, an omentum or an abdominal cavity.

23.-24. (canceled)

25. The method of claim 15, wherein the composition comprises about 60% to 94% of human collagen I, about 4% to 15% of human albumin, about 0.4% to 4% of L-glutamine, and about 1% to 5% of NaHCO₃.

26.-28. (canceled)

29. A pharmaceutical composition comprising collagen I, albumin, L-glutamine, NaHCO₃ and autologous β-cells of pancreatic islets of a subject.

30. The pharmaceutical composition of claim 29, wherein the autologous β-cells are stem cell-derived β-cells.

31.-32. (canceled)

33. The pharmaceutical composition of claim 29, wherein the composition comprises about 60% to 94% of human collagen I, about 4% to 15% of human albumin, about 0.4% to 4% of L-glutamine, and about 1% to 5% of NaHCO₃.

34.-36. (canceled)

37. A method of treating Type I diabetes in a subject in need thereof, comprising: administering the pharmaceutical composition of claim 29 to a non-percutaneous trans-hepatic site of the subject.

38. The method of claim 37, wherein the non-percutaneous trans-hepatic site is a subcutaneous space, a retroperitoneal space, an omentum or an abdominal cavity.

39. The method of claim 37, wherein the subject is a human or a non-human animal.

40. The method of claim 37, wherein the autologous β-cells are human β-cells, murine β-cells, or porcine β-cells.

41.-43. (canceled)

44. The method of claim 37, wherein the pharmaceutical composition comprises about 60% to 94% of human collagen I, about 4% to 15% of human albumin, about 0.4% to 4% of L-glutamine, and about 1% to 5% of NaHCO₃.

45.-47. (canceled)

48. A pharmaceutical composition comprising collagen I, albumin, L-glutamine, NaHCO₃ and autologous cells of a subject.

49. The pharmaceutical composition of claim 48, wherein the autologous cells are endocrine and/or secretory cells selected from the group consisting of hepatocytes, parathyroid cells and adrenal gland cells.

50. The pharmaceutical composition of claim 48, wherein the endocrine cells are stem cell-derived endocrine cells.

51.-52. (canceled)

53. The pharmaceutical composition of claim 48, wherein the composition comprises about 60% to 94% of human collagen I, about 4% to 15% of human albumin, about 0.4% to 4% of L-glutamine, and about 1% to 5% of NaHCO₃.

54.-56. (canceled)

57. A method of treating acute liver failure or hepatitis in a subject in need thereof, comprising: administering the pharmaceutical composition of claim 48 to a non-percutaneous trans-hepatic site of the subject, wherein the autologous cells are hepatocytes.

58. The method of claim 57, wherein the non-percutaneous trans-hepatic site is a subcutaneous space, a retroperitoneal space, an omentum or an abdominal cavity.

59. The method of claim 57, wherein the autologous hepatocytes are stem cell-derived hepatocytes.

60.-66. (canceled)
